(19) United States
(12) Patent Application Publication
(10) Pub. No.: US 2004/0230229 A1
(43) Pub. Date: Nov. 18, 2004

(54) Hybrid transthoracic/intrathoracic cardiac stimulation devices and methods

(76) Inventors: Eric G. Lovett, Roseville, MN (US); Mike Favet, Vadnais Heights, MN (US); Adam W. Cates, Minneapolis, MN (US); Kristine M. Larsen-Kelly, Lino Lakes, MN (US); Paul Haefner, Circle Pines, MN (US); F. Roosevelt Gilliam III, Durham, NC (US)

Correspondence Address: Crawford Maunu PLLC, Suite 390, 1270 Northland Drive, St. Paul, MN, 55120, US

(21) Appl. No.: 10/462,001

(22) Filed: Jun. 13, 2003

Related U.S. Application Data
(60) Provisional application No. 60/462,272, filed on Apr. 11, 2003.

(51) Int. Cl. A61N 1/362

(52) U.S. Cl. 607/4

(57) ABSTRACT

A cardiac sensing and stimulation system includes a housing within which energy delivery circuitry and detection circuitry are provided. Subcutaneous electrodes are coupled to the energy delivery and detection circuitry and arranged in a non-contacting relationship with respect to cardiac tissue, great vessels, and coronary vasculature. A lead system is coupled to the energy delivery and detection circuitry. The lead system electrodes are configured to contact cardiac tissue, great vessels, or coronary vasculature. A controller, provided in the housing, is coupled to the energy delivery and detection circuitry. The controller configures the system to operate in a first mode using at least the subcutaneous electrodes, and to operate in a second mode using at least the lead electrodes. The controller can selectively switch between the first and second modes, and selectively enable and disable components and circuitry associated with the first and second modes and combinations of these modes.

### RELATED APPLICATIONS

This application claims the benefit of Provisional Patent Application Serial No. 60/462,272, filed on Apr. 11, 2003, to which priority is claimed pursuant to 35 U.S.C. §119(e) and which is hereby incorporated herein by reference.

### FIELD OF THE INVENTION

The present invention relates generally to implantable medical devices and, more particularly, to methods and systems that provide for transthoracic, intrathoracic, and combined transthoracic/intrathoracic cardiac sensing and stimulation.

### BACKGROUND OF THE INVENTION

The healthy heart produces regular, synchronized contractions. Rhythmic contractions of the heart are normally controlled by the sinoatrial (SA) node, which are specialized cells located in the upper right atrium. The SA node is the normal pacemaker of the heart, typically initiating 60-100 heart beats per minute. When the SA node is pacing the heart normally, the heart is said to be in normal sinus rhythm.

If the heart's electrical activity becomes uncoordinated or irregular, the heart is denoted to be arrhythmic. Cardiac arrhythmia impairs cardiac efficiency and can be a potential life threatening event. Cardiac arrhythmias have a number of etiological sources, including tissue damage due to myocardial infarction, infection, or degradation of the heart's ability to generate or synchronize the electrical impulses that coordinate contractions.

Bradycardia occurs when the heart rhythm is too slow. This condition may be caused, for example, by impaired function of the SA node, denoted sick sinus syndrome, or by delayed propagation or blockage of the electrical impulse between the atria and ventricles. Bradycardia produces a heart rate that is too slow to maintain adequate circulation.

When the heart rate is too rapid, the condition is denoted tachycardia. Tachycardia may have its origin in either the atria or the ventricles. Tachycardias occurring in the atria of the heart, for example, include atrial fibrillation and atrial flutter. Both conditions are characterized by rapid contractions of the atria. Besides being hemodynamically inefficient, the rapid contractions of the atria can also adversely affect the ventricular rate.

Ventricular tachycardia, for example, occurs when electrical activity arises in the ventricular myocardium at a rate more rapid than the normal sinus rhythm. Ventricular tachycardia can quickly degenerate into ventricular fibrillation. Ventricular fibrillation is a condition denoted by extremely rapid, uncoordinated electrical activity within the ventricular tissue. The rapid and erratic excitation of the ventricular tissue prevents synchronized contractions and impairs the heart's ability to effectively pump blood to the body, which is a fatal condition unless the heart is returned to sinus rhythm within a few minutes.

Implantable cardioverter/defibrillators (ICDs) have been used as an effective treatment for patients with serious cardiac arrhythmias. Such ICDs are capable of delivering high energy shocks to the heart, interrupting the ventricular tachyarrythmia or ventricular fibrillation, and allowing the heart to resume normal sinus rhythm. ICDs may also include pacing functionality.

For reasons stated above, and for other reasons which will become apparent to those skilled in the art upon reading the present specification, there is a need for systems and methods that provide for enhanced sensing and therapy delivery capabilities. There remains a continuing need for safe and effective therapies for treating a variety of cardiac arrhythmias in a greater range of patient populations. There is yet a further need for systems and methods that facilitate research and development of new and alternative cardiac sensing, detection, and therapy delivery approaches. The present invention fulfills these and other needs, and addresses deficiencies in known systems and techniques.

### SUMMARY OF THE INVENTION

The present invention is directed to cardiac sensing and stimulation methods and systems. The present invention is particularly directed to methods and systems that provide for transthoracic, intrathoracic, and combined transthoracic/intrathoracic cardiac sensing and stimulation.

In accordance with one embodiment of the present invention, a cardiac sensing and stimulation system includes a housing within which energy delivery circuitry and detection circuitry are provided. One or more subcutaneous electrodes are coupled to the energy delivery and detection circuitry and arranged in a non-contacting relationship with respect to cardiac tissue, great vessels, and coronary vasculature. A lead system, comprising one or more lead electrodes, is coupled to the energy delivery and detection circuitry. The lead electrodes are configured to contact cardiac tissue, great vessels, or coronary vasculature.

A controller, provided in the housing, is coupled to the energy delivery and detection circuitry. The controller configures the system to operate in a first mode using at least the subcutaneous electrodes, and to operate in a second mode using at least the lead electrodes. The controller can selectively switch between the first and second modes, and selectively enable and disable components and circuitry associated with the first and second modes. For example, the first mode can define a transthoracic mode and the second mode can define an intrathoracic mode. The controller can selectively enable and disable these modes, and configure the system to operate using a combination of transthoracic and intrathoracic components and circuitry.

According to another embodiment, the system is configurable by the controller to operate in a standard of care configuration, using at least the lead electrodes, and in an alternative or test configuration, using at least the subcutaneous electrodes. Each of the standard of care and alternative system configurations is capable of providing cardiac activity sensing and stimulation in an independent or cooperative manner.

In one embodiment, a first system of a multiple system device is configured as a standard of care system. A second system of the multiple system device is configured as a monitoring system. The monitoring system monitors performance of the standard of care system. The first or second system can be an intrathoracic system, and the other of the first and second systems can be a transthoracic system, for example.

In accordance with a further embodiment, the controller of the above-described system configures the system to perform a particular function when operating in each of the first and second modes and to acquire performance data associated with performance of the particular function when operating in each of the first and second modes. For example, the particular function subject to evaluation can be a function associated with bradycardia and tachycardia sensing, a function associated with tachyarrhythmia detection or treatment, a function associated with one or both of stimulus waveform generation and stimulus waveform delivery, or a function involving a configuration of one or both of the lead system and the subcutaneous electrodes. The particular function subject to evaluation can also comprise a first sub-function associated with rate-based tachyarrhythmia detection and a second sub-function associated with morphology-based tachyarrhythmia detection, for example.

According to another embodiment of the present invention, a method of cardiac sensing and stimulation involves transthoracicly sensing cardiac activity in a first mode and, in response to cardiac conditions necessitating therapy sensed while operating in the first mode, delivering cardiac stimulation therapy transthoracicly or intrathoracicly. The method also involves intrathoracicly sensing cardiac activity in a second mode and, in response to cardiac conditions necessitating therapy sensed while operating in the second mode, intrathoracicly or transthoracicly delivering cardiac stimulation therapy. The method further involves selectively enabling and disabling the first and second modes.

In another approach, cardiac activity is sensed transthoracicly or intrathoracicly in a first mode and, in response to cardiac conditions necessitating therapy sensed while operating in the first mode, cardiac stimulation therapy is delivered transthoracicly. Further to this approach, cardiac activity is sensed intrathoracicly or transthoracicly in a second mode and, in response to cardiac conditions necessitating therapy sensed while operating in the second mode, cardiac stimulation therapy is delivered intrathoracicly.

According to a further approach, cardiac activity is sensed transthoracicly in a first mode and, in response to cardiac conditions necessitating therapy sensed while operating in the first mode, cardiac stimulation therapy is delivered transthoracicly or intrathoracicly. Cardiac activity is sensed intrathoracicly in a second mode and, in response to cardiac conditions necessitating therapy sensed while operating in the second mode, cardiac stimulation therapy is delivered intrathoracicly or transthoracicly. A particular function is performed when operating in each of the first and second modes. Performance data associated with performance of the particular function when operating in each of the first and second modes is acquired for subsequent evaluation.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIG. 1 is a view of a hybrid transthoracic/intrathoracic cardiac stimulation device implanted in a patient in accordance with an embodiment of the present invention;

FIG. 2 is a view of a hybrid cardiac stimulation device implanted in a patient in accordance with another embodiment of the present invention;

FIG. 3 is a view of a multi-chamber hybrid cardiac stimulation device implanted in a patient's heart in accordance with an embodiment of the present invention;

FIG. 4 is a view of a multi-site dual-chamber hybrid cardiac stimulation device implanted in a patient's heart in accordance with an embodiment of the present invention;

FIG. 5 is a block diagram showing various components of an intrathoracic cardiac stimulation system of a hybrid transthoracic/intrathoracic cardiac stimulation device in accordance with an embodiment of the present invention;

FIG. 6 is a block diagram showing various components of a transthoracic cardiac stimulation system of a hybrid transthoracic/intrathoracic cardiac stimulation device in accordance with an embodiment of the present invention;

FIG. 7 is a block diagram illustrating various processing and detection components of a transthoracic cardiac stimulation system of a hybrid transthoracic/intrathoracic cardiac stimulation device in accordance with an embodiment of the present invention;

FIG. 8 is a block diagram showing various sensors, devices, and circuitry of a hybrid transthoracic/intrathoracic cardiac stimulation device in accordance with an embodiment of the present invention;

FIG. 9 is a flow diagram illustrating various processes associated with multiple mode operation of a hybrid transthoracic/intrathoracic cardiac stimulation device in accordance with an embodiment of the present invention;

FIG. 10 is a flow diagram illustrating various processes associated with multiple configuration selection by a hybrid transthoracic/intrathoracic cardiac stimulation device in accordance with an embodiment of the present invention;

FIG. 11 is a flow diagram illustrating various manners by which multiple mode processes of a hybrid transthoracic/intrathoracic cardiac stimulation device can be effected in accordance with an embodiment of the present invention;

FIG. 12 is a flow diagram illustrating various processes associated with a particular multiple mode operation of a hybrid transthoracic/intrathoracic cardiac stimulation device in accordance with an embodiment of the present invention;

FIG. 13 is a flow diagram illustrating various processes associated with evaluating performance of a multiple mode hybrid transthoracic/intrathoracic cardiac stimulation device in accordance with an embodiment of the present invention;

FIGS. 14A and 14B are flow diagrams illustrating two approaches to monitoring hybrid cardiac stimulation device performance in accordance with an embodiment of the present invention;

FIG. 15 is a flow diagram illustrating a process by which performance of a first function by a first system of a hybrid transthoracic/intrathoracic cardiac stimulation device is enhanced by performance of a second function by a second system of the hybrid cardiac stimulation device in accordance with an embodiment of the present invention;

FIG. 16 is a flow diagram illustrating a process by which atrial therapy is provided by a first system of a hybrid transthoracic/intrathoracic cardiac stimulation device and ventricular tachyarrythmia backup therapy is provided by a second system of the hybrid cardiac stimulation device in accordance with an embodiment of the present invention;

FIG. 17 is a flow diagram illustrating various processes associated with detecting and treating ventricular fibrillation through cooperative operation of multiple systems of a hybrid transthoracic/intrathoracic cardiac stimulation device in accordance with an embodiment of the present invention; and

FIG. 18 illustrates a flow diagram illustrating various processes involving a cross-over study conducted for a given patient population using a transthoracic/intrathoracic cardiac stimulation device of the present invention implanted in each patient of the population.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings which form a part hereof, and in which is shown by way of illustration, various embodiments in which the invention may be practiced. It is to be understood that other embodiments may be utilized, and structural and functional changes may be made without departing from the scope of the present invention.

An implantable cardiac device implemented in accordance with the principles of the present invention can include one or more of the features, structures, methods, or combinations thereof described below and in the above-identified Provisional Application. For example, a cardiac stimulator or monitor can be implemented to include one or more of the advantageous features and/or processes described below and in the above-identified Provisional Application. It is intended that such a stimulator, monitor or other implanted or partially implanted device need not include all of the features described herein, but can be implemented to include selected features that provide for unique structures and/or functionality.

One such device, termed an implantable hybrid transthoracic/intrathoracic cardiac stimulation device (hybrid device), is described herein to include various advantageous features and/or processes. It is understood that the description of features and processes within the context of a hybrid device is provided for non-limiting illustrative purposes only, and that such features and process can be implemented in other types of devices, including implantable and non-implantable devices. For example, features and processes described herein can be implemented in cardiac monitors, pacemakers, cardioverters/defibrillators, resynchronizers, and the like, including those devices disclosed in the various patents incorporated herein by reference. It is further understood that features and processes described herein can be implemented in devices that use one or more of transvenous, endocardial, epicardial, subcutaneous or surface electrodes, or devices that use combinations of these electrodes.

A significant challenge to development of cardiac rhythm management (CRM) or cardiac function management (CFM) systems is the collection of information that verifies proper function of the system. At present, a conventional transvenous configuration is generally preferred for both pacemakers and defibrillators. This approach superceded previous configurations (e.g., epicardial) only after significant clinical work to demonstrate comparable safety and efficacy.

In contrast to conventional transvenous system configurations, a hybrid approach of the present invention employs an implantable device that supports at least two independent but integrated cardiac stimulation systems. In general, each system typically includes sensing, detection, diagnostics, and therapy capabilities, although one or both of the systems may provide minimal capabilities in less sophisticated configurations, such as sensing or monitoring capabilities.

According to one embodiment, one system of a hybrid device can be implemented in accordance with a conventional transvenous based electrode configuration (which can include one or more of transvenous, endocardial, and/or epicardial electrodes), and a second system of the hybrid device can be implemented as a subcutaneous-only system. Alternatively, one system can be implemented as a standard of care system, while the other is implemented as a test system. It is understood that a hybrid device can be implemented to include three or more independent but integrated cardiac monitoring and/or stimulation devices.

The systems of a hybrid device can operate simultaneously (in parallel), tiered (e.g., in the same arrhythmic episode) or sequentially. The hybrid device, for example, can alternate between conventional and subcutaneous configurations or modes in a predetermined manner. For example, the hybrid device can operate in a conventional configuration for N arrhythmic episodes, and then switch to a subcutaneous configuration for M arrhythmic episodes, where N can be equal to or different from M. Such configuration and mode switching can be dictated in accordance with system programming (i.e., firmware or software) or in response to command signals generated by an external device, such as a programmer.

A hybrid transthoracic/intrathoracic cardiac stimulation device can advantageously be used where it is desired to retain the benefit of conventional or widely approved cardiac rhythm management (CRM) while exploring new detection and therapy alternatives. For example, a hybrid approach of the present invention allows upgrading of therapy for patients who develop additional comorbidities, and allows for rapid development of novel cardiac management technologies. A hybrid approach can also provide proof of feasibility for new systems without sacrificing safety and efficacy that an established system provides. For example, a hybrid device can be used to facilitate development and introduction of subcutaneous defibrillation technologies, while providing conventional CRM support.

A hybrid approach can, for example, provide direct comparison of new versus established systems data (paired data). The co-system of the hybrid configuration, for example, can provide supplemental data that improves performance of the primary system (e.g., far-field signal from subcutaneous lead could improve rhythm diagnosis). In particular, a hybrid device can facilitate data collection and comparison of such data (by the hybrid device or by an external processing system) in a variety of ways for research and development, and in product design, implementation, and eventual use in the patient.

A hybrid device of the present invention is particularly well suited to facilitate development of subcutaneous cardiac rhythm management systems by permitting acquisition of crucial data from patients in chronic, ambulatory environments. Hybrid devices of the present invention provide for collection of experimental data with the safety of existing, market-approved technology. Such hybrid devices also permit the comparison of new and existing technologies in cross-over study designs, a valuable technique for collecting paired data. The implant procedure for hybrid devices provides an opportunity to acquire acute sensing/detection and/or therapy data as well as experience with leads, delivery systems, and surgical procedures associated with implant.

The functionality of conventional cardiac rhythm management devices can be significantly enhanced by addition of transthoracic sensing and/or stimulation capabilities in accordance with one hybrid device implementation approach. By way of example, a cardiac resynchronization therapy defibrillator (CRT-D) can provide cardiac resynchronization therapy for the treatment of heart failure by providing electrical stimulation to the right and left ventricles or left ventricle only to synchronize ventricular contractions. Such a device also provides ventricular tachyarrhythmia therapy to treat ventricular tachycardia (VT) and ventricular fibrillation (VF), rhythms that are associated with sudden cardiac death (SCD). A hybrid device can be configured to include CRT-D circuitry and modified to include a subcutaneous sensing lead, which can be connected to the left ventricular and/or right atrial sense channel (or other channel) of the CRT-D circuitry, for example. This hybrid device can be further modified to store and telemeter subcutaneous electrograms as appropriate. The remainder of the functionality can provide normal ICD-VR operation using transvenous leads.

In another approach, a hybrid device can be configured to include implantable cardioverter/defibrillator (ICD) circuitry that further provides for advanced atrial arrhythmia management. This hybrid device can include features designed to manage abnormal heart rates in the atrial and ventricular chambers of the heart. A hybrid device of this configuration can include capacitors, batteries, and high voltage components capable of delivering high voltage stimulation energy to the heart. For example, a hybrid device can be implemented to deliver up to 120 J, 1800V shocks.

In accordance with another implementation, a hybrid device incorporating ICD circuitry can be enhanced to include subcutaneous sensing and detection algorithms, and the capability to revise such algorithms after manufacture. Such a hybrid device can be programmed to compare subcutaneous and conventional sensing and detection effectiveness while running in parallel modes, for example. Subcutaneous or conventional sensing/detection can be used to determine device behavior based on programming. Therapy can be programmed to be exclusively intrathoracic, a blend of intrathoracic and transthoracic, or exclusively transthoracic.

Elements of a hybrid transthoracic/intrathoracic cardiac stimulation device can be implanted under the skin in the chest region of a patient. Elements of the hybrid device may, for example, be implanted subcutaneously such that selected elements of the device are positioned on the patient's front, back, side, or other body locations suitable for sensing cardiac activity and/or delivering cardiac stimulation therapy. It is understood that elements of the hybrid device may be located at several different body locations, such as in the chest, abdominal, or subclavian region, with electrode elements respectively positioned at different regions near, around, in, or on the heart. For example, intrathoracic lead/electrode elements of the hybrid device can be positioned on or within the heart, great vessel or coronary vasculature.

The primary housing (e.g., the active or non-active can) of the hybrid device, for example, can be configured for positioning outside of the rib cage at an intercostal or subcostal location, within the abdomen, or in the upper chest region (e.g., subclavian location, such as above the third rib). A transthoracic configuration of the hybrid device typically employs one or more electrodes located on, or extending from, the primary housing and/or at other locations about, but not in direct contact with, the heart, great vessel or coronary vasculature. Such electrodes are generally referred to herein as subcutaneous electrodes, it being understood that surface electrodes can also be employed in certain configurations. One or more subcutaneous electrode arrays, for example, can be used to sense cardiac activity and deliver cardiac stimulation energy in a hybrid device configuration employing an active can or a configuration employing a non-active can. Electrodes can be situated at anterior and/or posterior locations relative to the heart.

An intrathoracic configuration of the hybrid device typically employs one or more electrodes positioned in direct contact with the heart, great vessel or coronary vasculature. The intrathoracic electrodes are typically connected to the primary housing via one or more leads. The intrathoracic configuration of a hybrid device can employ one or more of transvenous or venous electrodes, endocardial electrodes, and epicardial electrodes.

A hybrid device of the present invention includes a controller or control system that can alter the configuration and operating modes of the device. For example, the controller can configure the hybrid device to operate in a standard of care configuration using at least the lead electrodes of the intrathoracic system, and to operate in a test or alternative configuration using at least the subcutaneous electrodes of the transthoracic system. The controller can also configure the hybrid device to use selected combinations of intrathoracic and transthoracic electrodes for operations associated with each of the various operating modes and/or individual functions or therapies.

Alterations in the operating configuration or mode of a hybrid device can be initiated and controlled in a variety of ways. For example, the hybrid device can switch operating modes or configurations in response to a configuration signal received from a patient-external signal source, such as from a programmer or patient/clinician controlled activator. The controller of a hybrid device can also change modes or configurations in response to a predetermined condition, such as unsuccessful detection of an arrhythmia, unsuccessful treatment of an arrhythmia, expiration of a predetermined amount of time, occurrence of a scheduled event, occurrence of a predetermined number of arrhythmic episodes, or occurrence of a predetermined type of arrhythmia, for example. Hybrid device mode or configuration switching can be effected to enhance sensing, detection, and/or therapy delivery operations, such as arrhythmia detection, treatment, and cessation confirmation. Such switching can involve selective enabling and disabling of the intrathoracic system, the transthoracic system, and particular components and functions of the respective intrathoracic and transthoracic systems.

Certain system configurations illustrated herein are generally described as capable of implementing various functions traditionally performed by an implantable cardioverter/defibrillator (ICD), and may operate in numerous cardioversion/defibrillation modes as are known in the art. Exemplary ICD circuitry, structures and functionality, aspects of which can be incorporated in a hybrid device of a type contemplated herein, are disclosed in commonly owned U.S. Pat. Nos. 5,133,353; 5,179,945; 5,314,459; 5,318,597; 5,620,466; and 5,662,688, which are hereby incorporated herein by reference in their respective entireties.

In particular configurations, systems and methods can perform functions traditionally performed by pacemakers, such as providing various pacing therapies as are known in the art, in addition to cardioversion/defibrillation therapies. Exemplary pacemaker circuitry, structures and functionality, aspects of which can be incorporated in a hybrid device of a type contemplated herein, are disclosed in commonly owned U.S. Pat. Nos. 4,562,841; 5,284,136; 5,376,106; 5,036,849; 5,540,727; 5,836,987; 6,044,298; and 6,055,454, which are hereby incorporated herein by reference in their respective entireties.

A hybrid device can implement functionality traditionally provided by cardiac monitors as are known in the art, in addition to providing cardioversion/defibrillation therapies. Exemplary cardiac monitoring circuitry, structures and functionality, aspects of which can be incorporated in a hybrid device of a type contemplated herein, are disclosed in commonly owned U.S. Pat. Nos. 5,313,953; 5,388,578; and 5,411,031, which are hereby incorporated herein by reference in their respective entireties.

A hybrid device may implement various anti-tachyarrhythmia therapies, such as tiered therapies, which may involve performing rate-based, pattern and rate-based, and/or morphological tachyarrhythmia discrimination analyses. Subcutaneous, cutaneous, and/or external sensors can be employed to acquire physiologic and non-physiologic information for purposes of enhancing tachyarrhythmia detection and termination. It is understood that configurations, features, and combination of features described in the instant disclosure can be implemented in a wide range of implantable medical devices, and that such embodiments and features are not limited to the particular devices described herein.

It is also understood that the components and functionality depicted in the figures and described herein can be implemented in hardware, software, or a combination of hardware and software. It is further understood that the components and functionality depicted as separate or discrete blocks/elements in the figures can be implemented in combination with other components and functionality, and that the depiction of such components and functionality in individual or integral form is for purposes of clarity of explanation, and not of limitation.

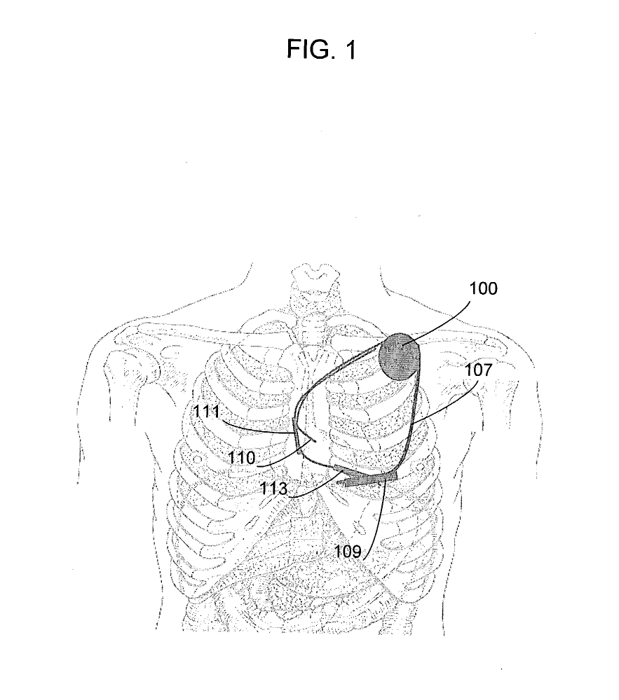

Referring now to FIG. 1 of the drawings, there is shown a configuration of a hybrid transthoracic/intrathoracic cardiac stimulation device implanted in the chest region of a patient in accordance with an embodiment of the present invention. A typical hybrid device configuration includes one or more subcutaneous electrodes and one or more transvenous, epicardial, and/or endocardial electrodes. A hybrid device, according to one configuration, can include a conventional (e.g., transvenous) system implemented together with investigational (e.g., subcutaneous) components. This configuration has the benefit of retaining the safety and efficacy of the conventional system while allowing evaluation of the investigational components.

With regard to the particular configuration shown in FIG. 1, the hybrid device includes a housing 100 within which various cardiac sensing, detection, processing, and energy delivery circuitry can be housed. Communications circuitry is disposed within the housing 100 for facilitating communication between the hybrid device and an external communication device, such as a portable or bed-side communication station, patient-carried/worn communication station, or external programmer, for example. The communications circuitry can also facilitate unidirectional or bidirectional communication with one or more external, cutaneous, or subcutaneous physiologic or non-physiologic sensors.

The housing 100 is typically configured to include one or more electrodes (e.g., can electrode and/or indifferent electrode). Although the housing 100 is typically configured as an active can, it is appreciated that a non-active can configuration may be implemented, in which case at least two electrodes spaced apart from the housing 100 are typically employed.

In the configuration shown in FIG. 1, a subcutaneous electrode 109 can be positioned under the skin in the chest region and situated distal from the housing 100. The subcutaneous and, if applicable, housing electrode(s) can be positioned about the heart at various locations and orientations, such as at various anterior and/or posterior locations relative to the heart. The subcutaneous electrode 109 is electrically coupled to circuitry within the housing 100 via a lead assembly 107. One or more conductors (e.g., coils or cables) are provided within the lead assembly 107 and electrically couple the subcutaneous electrode 109 with circuitry in the housing 100. One or more sense, sense/pace or defibrillation electrodes can be situated on the elongated structure of the electrode support, the housing 100, and/or the distal electrode assembly.

The hybrid device shown in FIG. 1 further includes an endocardial lead system, which is electrically coupled to circuitry within the housing 100 via one or more transvenous leads. The endocardial lead system is preferably implanted using a conventional transvenous lead delivery procedure. The endocardial lead system can include a single lead for implant within or to a single heart chamber (atrial or ventricular chamber) or multiple heart chambers (e.g., single pass lead). More than one lead can be deployed (e.g., right and/or left heart leads) for implant within one or multiple heart chambers (e.g., multisite or multi-chamber configuration). As such, a hybrid device can be implanted to provide intrathoracic sensing and/or stimulation therapy in one, two, three, or four heart chambers.

In FIG. 1, an atrial lead system includes a lead (e.g., right atrial lead) for electrically coupling the housing circuitry with one or more atrial electrodes 110. A ventricular defibrillation lead system can include one or two leads for electrically coupling the housing circuitry with one or more ventricular electrodes. The ventricular defibrillation lead system can include, for example, a right ventricular electrode 113 and an electrode 111 positioned in the superior vena cava.

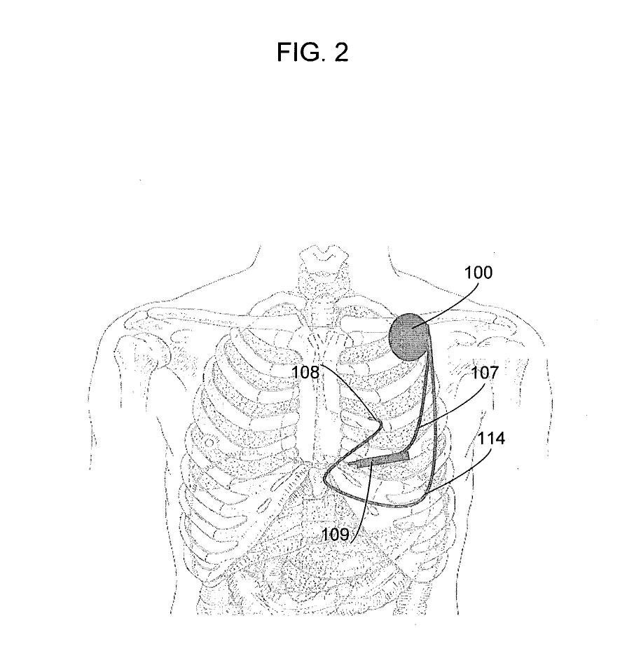

The hybrid device shown in FIG. 2 includes the subcutaneous electrode and housing components shown in FIG. 1, but employs one or more epicardial or transvenous lead systems instead of the endocardial lead approach shown in FIG. 1. A typical transvenous lead system can include one or more electrodes adapted for implant within a great vessel (e.g., coronary or pulmonary vessel) or coronary vasculature. A typical epicardial lead system can include one or more patch-type and/or screw-in electrodes or other electrode configuration that contacts the epicardium of the heart.

In FIG. 2, an intrathoracic lead 114 includes one or more distal electrodes 108 that can be configured for epicardial or transvenous cardiac activity sensing and/or stimulation energy delivery. As shown, a single lead 114 electrically couples the intrathoracic electrode(s) 108 with circuitry provided in the housing 100. It is appreciated that one or more intrathoracic leads 114 can be deployed to provide sensing and stimulation energy delivery for one or more chambers of the heart.

In one configuration of the transthoracic portion of a hybrid device, the lead assembly 107 is generally flexible and has a construction similar to conventional implantable, medical electrical leads (e.g., defibrillation leads or combined defibrillation/pacing leads). In another configuration, the lead assembly 107 is constructed to be somewhat flexible, yet has an elastic, spring, or mechanical memory that retains a desired configuration after being shaped or manipulated by a clinician. For example, the lead assembly 107 can incorporate a gooseneck or braid system that can be distorted under manual force to take on a desired shape. In this manner, the lead assembly 107 can be shape-fit to accommodate the unique anatomical configuration of a given patient, and generally retains a customized shape after implantation. Shaping of the lead assembly 107 according to this configuration can occur prior to, and during, hybrid device implantation.

In accordance with a further configuration, the lead assembly 107 includes a rigid electrode support assembly, such as a rigid elongated structure that positionally stabilizes the subcutaneous electrode 109 with respect to the housing 100. In this configuration, the rigidity of the elongated structure maintains a desired spacing between the subcutaneous electrode 109 and the housing 100, and a desired orientation of the subcutaneous electrode 109/housing 100 relative to the patient's heart. The elongated structure can be formed from a structural plastic, composite or metallic material, and comprises, or is covered by, a biocompatible material. Appropriate electrical isolation between the housing 100 and subcutaneous electrode 109 is provided in cases where the elongated structure is formed from an electrically conductive material, such as metal.

In one configuration, the rigid electrode support assembly and the housing 100 define a unitary structure (i.e., a continuous housing/unit). The electronic components and electrode conductors/connectors are disposed within or on the unitary hybrid device housing/electrode support assembly. At least two electrodes are supported on the unitary structure, typically at or near opposing ends of the housing/electrode support assembly. The unitary structure can have an arcuate or angled shape, for example.

According to another configuration, the rigid electrode support assembly defines a physically separable unit relative to the housing 100. The rigid electrode support assembly includes mechanical and electrical couplings that facilitate mating engagement with corresponding mechanical and electrical couplings of the housing 100. For example, a header block arrangement can be configured to include both electrical and mechanical couplings that provide for mechanical and electrical connections between the rigid electrode support assembly and housing 100. The header block arrangement can be provided on the housing 100 or the rigid electrode support assembly. Alternatively, a mechanical/electrical coupler can be used to establish mechanical and electrical connections between the rigid electrode support assembly and housing 100. In such a configuration, a variety of different electrode support assemblies of varying shapes, sizes, and electrode configurations can be made available for physically and electrically connecting to a standard hybrid device housing 100.

It is noted that the electrodes and the lead assembly 107 can be configured to assume a variety of shapes. For example, the lead assembly 107 can have a wedge, chevron, flattened oval, or ribbon shape, and the subcutaneous electrode 109 can comprise a number of spaced electrodes, such as an array or band of electrodes. Moreover, two or more subcutaneous electrodes 109 can be mounted to multiple electrode support assemblies 107 to achieve a desired spaced relationship amongst subcutaneous electrodes 109. A hybrid device can incorporate circuitry, structures and functionality of the subcutaneous implantable medical devices disclosed in commonly owned U.S. Pat. Nos. 5,203,348; 5,230,337; 5,360,442; 5,366,496; 5,397,342; 5,391,200; 5,545,202; 5,603,732; and 5,916,243, which are hereby incorporated herein by reference in their respective entireties.

Depending on the configuration of a particular hybrid device, a delivery system can advantageously be used to facilitate proper placement and orientation of the hybrid device housing and subcutaneous electrode(s). According to one configuration of such a delivery system, a long metal rod similar to conventional trocars can be used to perform small diameter blunt tissue dissection of the subdermal layers. This tool may be pre-formed straight or curved to facilitate placement of the subcutaneous electrode, or it may be flexible enough to allow the physician to shape it appropriately for a given patient.

The tool can further include one or more fluid delivery channels and distal end perforations or ports to facilitate delivery of a local anesthetic continuously and accurately during tissue dissection to reduce/eliminate discomfort to a nonsedated or minimally sedated patient. A blunt tissue dissection tool can also be implemented to provide electrical stimulation for pain relief during blunt dissection. The dissection tool can be configured to include an energy delivery capability to provide stimulation similar to that provided by a TENS (transcutaneous nerve stimulation) unit. The energy delivered by the blunt tissue dissection tool essentially jams the nerve conduction by stimulating it with high frequency electrical stimulation. Exemplary delivery tools, aspects of which can be incorporated into a hybrid device delivery tool, are disclosed in the previously incorporated U.S. Provisional Application 60/462,272 and in commonly owned U.S. Pat. No. 5,300,106, which is hereby incorporated herein by reference in -its entirety.

In accordance with one embodiment, a hybrid transthoracic/intrathoracic cardiac stimulation device of the present invention can be configured to provide cardiac function management for patients suffering from heart failure. Heart failure is often associated with prolonged ventricular conduction delay, such as left bundle branch block, which contributes to left ventricular systolic dysfunction and poor outcome. Ventricular conduction delay generates uncoordinated ventricular contractions that reduce pumping effectiveness. Studies of heart failure patients in normal sinus rhythm with left ventricular conduction delay indicate that atrio-biventricular pacing can improve systolic function and pumping efficiency. Biventricular pacing may resynchronize right and left ventricular contractions as well as left ventricular septal and lateral wall contractions.

Another application of biventricular pacing involves correcting the left ventricular contraction delay induced by pacing only the right ventricle which reduces contractile function, cardiac output, and cardiac metabolic efficiency. When cardiac function is already depressed by heart disease, such as dilated cardiomyopathy or atrial fibrillation, further decline in heart function from right ventricular pacing may not be tolerated and may contribute to worsening symptoms and failure progression.

A hybrid device of the present invention can be configured to provide multichamber or multisite pacing for treatment of contractile dysfunction, while concurrently treating bradycardia and tachycardia. A hybrid device of this configuration can operate as a cardiac function management device, or CFM device, to improve pumping function by altering heart chamber contraction sequences while maintaining pumping rate and rhythm. Various CFM system configurations and functionality suitable for incorporation in a hybrid device of the present invention are disclosed in commonly owned U.S. patent application Ser. No. 10/270,035, filed Oct. 11, 2002 under Attorney Docket No. GUID.049PA, which is hereby incorporated herein by reference.

A hybrid transthoracic/intrathoracic cardiac stimulation device incorporating a multichamber pacemaker may include electrodes positioned to contact cardiac tissue within or adjacent to both the left and the right ventricles for pacing both the left and right ventricles. Furthermore, pacemaker circuitry of the hybrid device may be coupled to electrodes positioned to contact tissue within or adjacent to both the left and the right atria to enable bi-atrial pacing. Bi-atrial or bi-ventricular pacing may be used to improve the coordination of cardiac contractions between the bilateral heart chambers. Furthermore, a hybrid device may incorporate multisite pacemaker circuitry, which may be coupled to leads positioned in or adjacent to a heart chamber and positioned appropriately to pace two sites of the heart chamber.

Embodiments of a hybrid device that provide cardiac function management (CFM) may operate in numerous pacing modes. In one embodiment, a hybrid device configured as a multichamber defibrillator and pacemaker operates to stimulate the heart by delivering pace pulses according to various multichamber or multisite pacing timing modes. Many types of multiple chamber pacemaker/defibrillator methodologies may be used to implement the multichamber pacing modes according to this embodiment. Although the present hybrid device embodiment is described in conjunction with a CFM device implementation having a microprocessor-based architecture, it will be understood that the CFM device functionality may be implemented in any logic-based architecture, if desired.

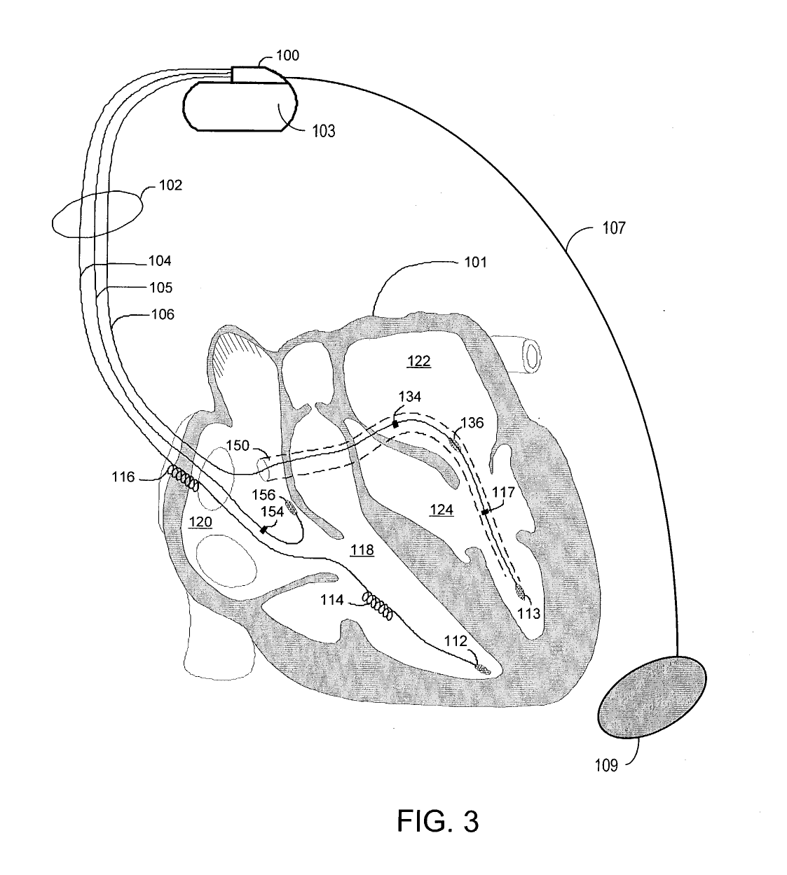

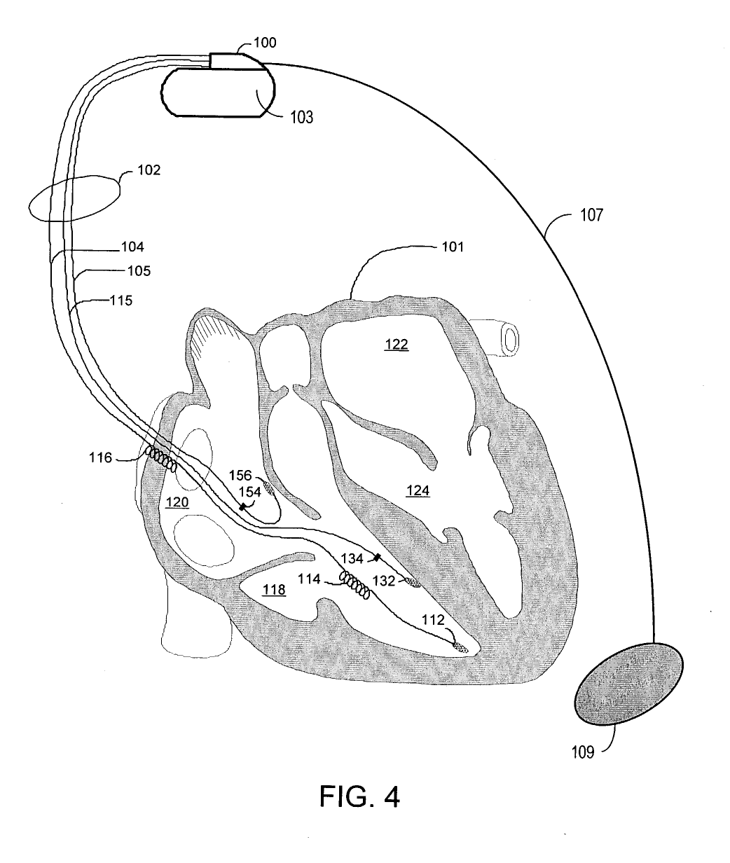

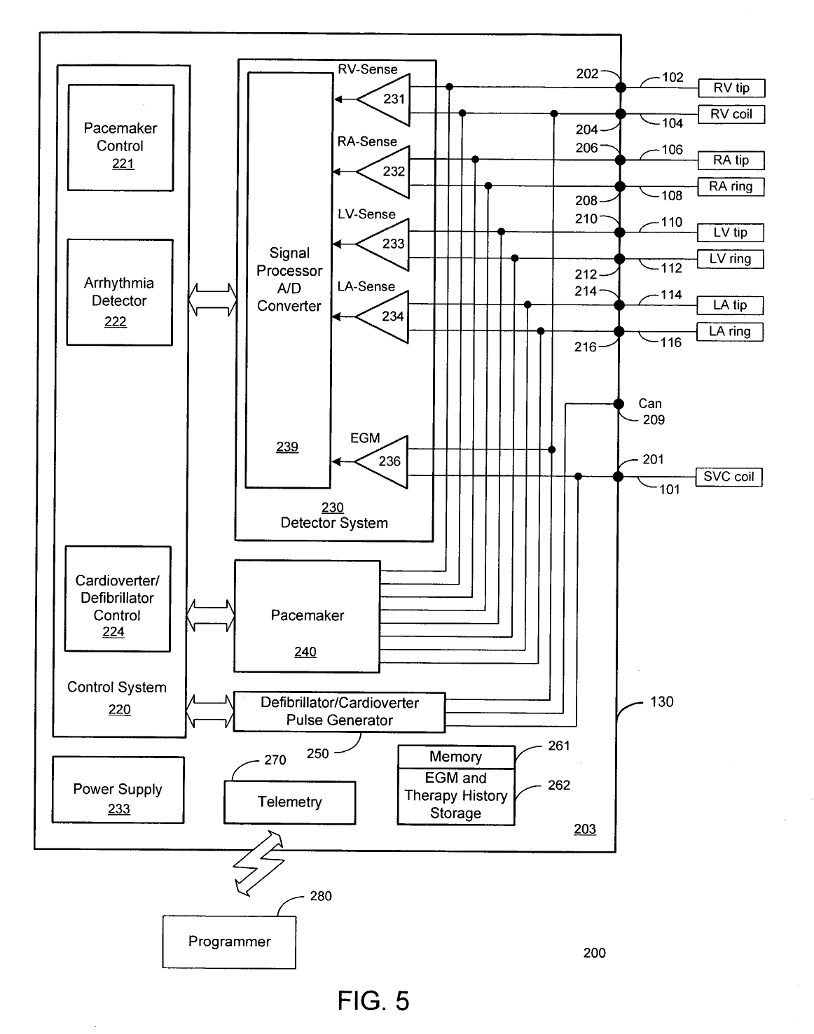

Referring now to FIG. 3 of the drawings, there is shown an embodiment of a hybrid transthoracic/intrathoracic cardiac stimulation device which incorporates CFM capabilities. It is understood that the system shown in FIG. 3 and related FIGS. 4 and 5 can be configured to perform conventional pacemaker and/or cardioversion/defibrillator functions in addition to, or to the exclusion of, CFM functions. The hybrid device includes a housing 100 electrically and physically coupled to an intracardiac lead system 102. The intracardiac lead system 102 is implanted in a human body with portions of the intracardiac lead system 102 inserted into a heart 101. The intracardiac lead system 102 is used to detect and analyze electric cardiac signals produced by the heart 101 and to provide electrical energy to the heart 101 under certain predetermined conditions to treat cardiac arrhythmias.

The intracardiac lead system 102 includes one or more electrodes used for pacing, sensing, or defibrillation. In the particular embodiment shown in FIG. 3, the intracardiac lead system 102 includes a right ventricular lead system 104, a right atrial lead system 105, and a left atrial/ventricular lead system 106. In one embodiment, the right ventricular lead system 104 is configured as an integrated bipolar pace/shock lead.

The right ventricular lead system 104 includes an SVC-coil 116, an RV-coil 114, and an RV-tip electrode 112. The RV-coil 114, which may alternatively be configured as an RV-ring electrode, is spaced apart from the RV-tip electrode 112, which is a pacing electrode for the right ventricle.

The right atrial lead system 105 includes a RA-tip electrode 156 and an RA-ring electrode 154. The RA-tip 156 and RA-ring 154 electrodes may provide pacing pulses to the right atrium of the heart and detect cardiac signals from the right atrium. In one configuration, the right atrial lead system 105 is configured as a J-lead.

In this configuration, the intracardiac lead system 102 is shown positioned within the heart 101, with the right ventricular lead system 104 extending through the right atrium 120 and into the right ventricle 118. In particular, the RV-tip electrode 112 and RV-coil electrode 114 are positioned at appropriate locations within the right ventricle 118. The SVC-coil 116 is positioned at an appropriate location within the right atrium chamber 120 of the heart 101 or a major vein leading to the right atrium chamber 120 of the heart 101. The RV-coil 114 and SVC-coil 116 depicted in FIG. 3 are defibrillation electrodes.

An LV-tip electrode 113, and an LV-ring electrode 117 are inserted through the coronary venous system and positioned adjacent to the left ventricle 124 of the heart 101. The LV-ring electrode 117 is spaced apart from the LV-tip electrode 113, which is a pacing electrode for the left ventricle. Both the LV-tip 113 and LV-ring 117 electrodes may also be used for sensing the left ventricle, thereby providing two sensing sites within the left ventricle. The left atrial/left ventricular lead system 106 further includes two LA-ring electrodes, LA-ring1 136 LA-ring2 134, positioned adjacent the left atrium 122 for pacing and sensing the left atrium 122 of the heart 101.

The left atrial/left ventricular lead system 106 includes endocardial pacing leads that are advanced through the superior vena cava (SVC), the right atrium 120, the valve of the coronary sinus, and the coronary sinus 150 to locate the LA-ring1 136, LA-ring2 134, LV-tip 113 and LV-ring 117 electrodes at appropriate locations adjacent to the left atrium and ventricle 122,124, respectively.

According to one lead delivery approach, left atrial/ventricular lead placement involves creating an opening in a percutaneous access vessel, such as the left subclavian or left cephalic vein. The left atrial/left ventricular lead 106 is guided into the right atrium 120 of the heart via the superior vena cava. From the right atrium 120, the left atrial/left ventricular lead system 106 is deployed into the coronary sinus ostium, the opening of the coronary sinus 150. The lead system 106 is guided through the coronary sinus 150 to a coronary vein of the left ventricle 124. This vein is used as an access pathway for leads to reach the surfaces of the left atrium 122 and the left ventricle 124 which are not directly accessible from the right side of the heart.

Lead placement for the left atrial/left ventricular lead system 106 may be achieved via the subclavian vein access and a preformed guiding catheter for insertion of the LV and LA electrodes 113, 117, 136, 134 adjacent the left ventricle 124 and left atrium 122, respectively. In one configuration, the left atrial/left ventricular lead system 106 is implemented as a single-pass lead.

FIG. 4 shows one embodiment of a hybrid device that may be used for synchronized multisite sensing or pacing within a heart chamber. The hybrid device includes a housing 100 electrically and physically coupled to an intracardiac lead system 102. The intracardiac lead system 102 includes one or more electrodes used for pacing, sensing, or defibrillation. In the particular embodiment shown in FIG. 4, the intracardiac lead system 102 includes first and second right ventricular lead systems 104, 115 and a right atrial lead system 105. In one embodiment, the right ventricular lead system 104 is configured as an integrated bipolar pace/shock lead.

The first right ventricular lead system 104 includes an SVC-coil 116, an RV-coil 114, and an RV-tip electrode 112. The RV-coil 114, which may alternatively be configured as an RV-ring electrode, is spaced apart from the RV-tip electrode 112, which is a pacing electrode for the right ventricle. The first right ventricular lead system includes endocardial pacing leads that are advanced through the superior vena cava (SVC), the right atrium 120 and into the right ventricle 118 to contact myocardial tissue at a first pacing site within the right ventricle 118.

The second right ventricular lead system 115 includes an RV-tip electrode 132 and an RV-ring electrode 134. The first right ventricular lead system 104 includes endocardial pacing leads that are advanced through the superior vena cava (SVC), the right atrium 120 and into the right ventricle 118 to contact myocardial tissue at a second pacing site within the right ventricle 118.

The right atrial lead system 105 includes a RA-tip electrode 156 and an RA-ring electrode 154. The RA-tip 156 and RA-ring 154 electrodes may provide respectively pacing pulses to the right atrium of the heart and detect cardiac signals from the right atrium. In one configuration, the right atrial lead system 105 is configured as a J-lead.

In this configuration, the intracardiac lead system 102 is shown positioned within the heart 101, with the first and the second right ventricular lead systems 104, 115 extending through the right atrium 120 and into the right ventricle 118. In particular, the RV-tip electrode 112 and RV-coil electrode 114 are positioned at appropriate locations to sense and pace a first site within the right ventricle 118. The SVC-coil 116 is positioned at an appropriate location within the right atrium chamber 120 of the heart 101 or a major vein leading to the right atrium chamber 120 of the heart 101. The RV-coil 114 and SVC-coil 116 depicted in FIG. 4 are defibrillation electrodes. An RV-tip electrode 132, and an RV-ring electrode 134 are positioned at appropriate locations to sense and pace a second site within the right ventricle 118.

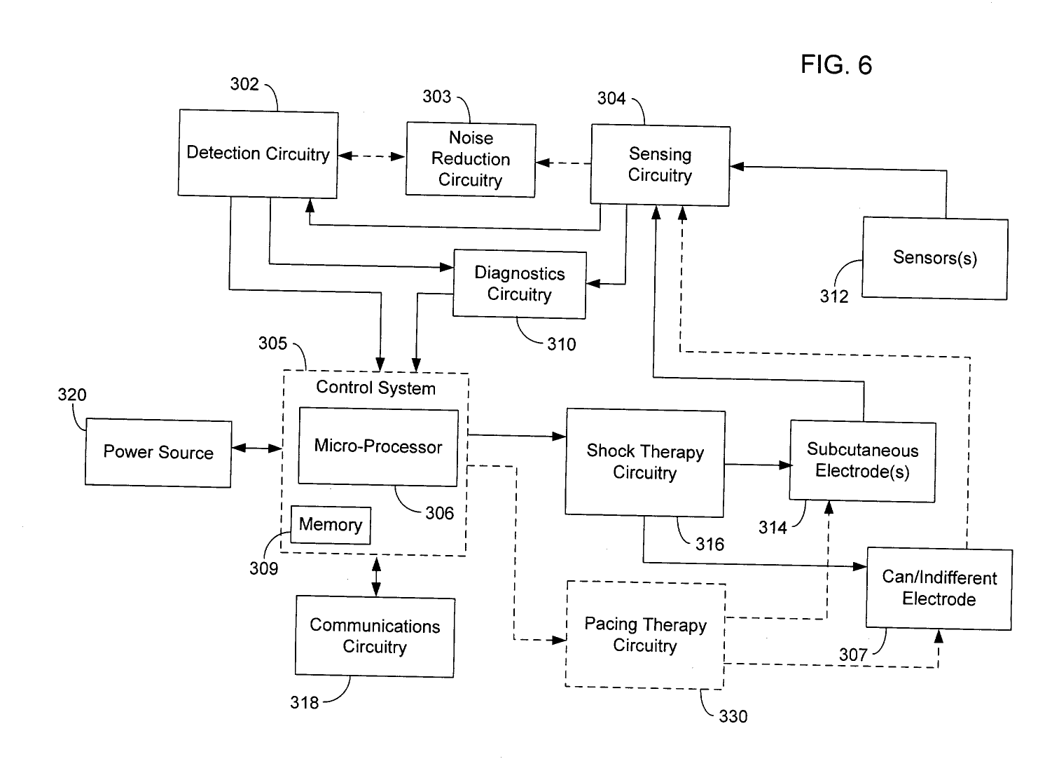

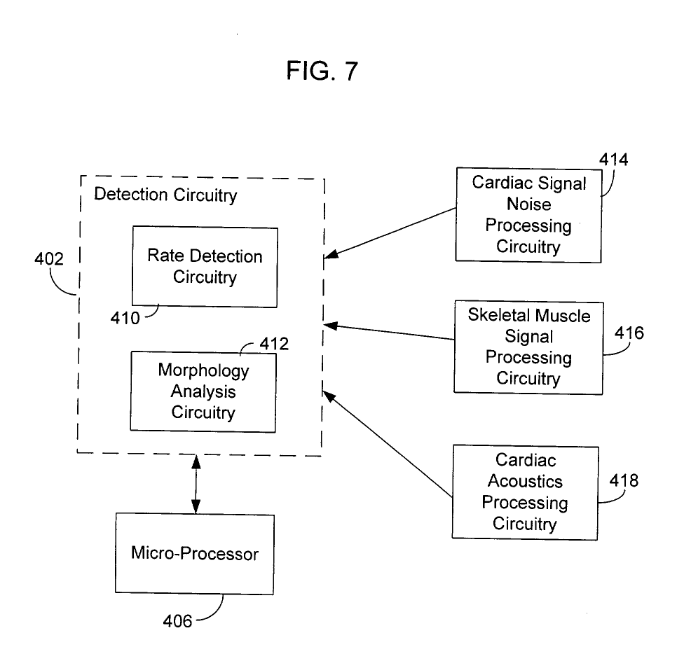

Referring now to FIG. 5, there is shown an embodiment of an intrathoracic system which may be incorporated within a hybrid transthoracic/intrathoracic cardiac stimulation device of the present invention. FIGS. 6 and 7 illustrate an embodiment of a transthoracic system which may be incorporated within a hybrid transthoracic/intrathoracic cardiac stimulation device. Although a hybrid device of the present invention incorporates components and functionality provided by both intrathoracic and transthoracic systems, such components and functionality are presented in separate figures for purposes of simplicity and clarity.

Moreover, it is understood that the embodiments depicted in FIGS. 5-8 may share similar components, and that such components can be implemented using a common component or implemented as separate components. Further, the embodiments depicted in FIGS. 5-8 may share similar functions, and that such functions can be implemented using a common approach or separate approach. For example, the circuitry shown in FIG. 5 includes a control system 220, which may be the same or different system as that shown as a control system 305 in FIG. 6.

The system 200 shown in FIG. 5 is suitable for implementing timing cycles for synchronized pacing in accordance with various embodiments of the present invention, including CFM embodiments. For purposes of illustration, the intrathoracic system 200 depicted in FIG. 5 will be described as having CFM functionality. The system 200 shown in FIG. 5 is divided into functional blocks. There exist many possible configurations in which these functional blocks can be arranged. The configuration depicted in FIG. 5 is one possible functional arrangement. The system 200 includes circuitry for receiving cardiac signals from a heart and delivering electrical energy in the form of pace pulses or cardioversion/defibrillation pulses to the heart.

The right ventricular lead system includes conductors 102 and 104 for transmitting sense and pacing signals between terminals 202 and 204 of the hybrid device and the RV-tip and RV-coil electrodes, respectively. The right ventricular lead system further includes conductor 101 for transmitting signals between the SVC coil and terminal 201 of the hybrid device. The right atrial lead system includes conductor 106 for transmitting signals between the RA-tip electrode and terminal 206 and conductor 108 for transmitting signals between the RA-ring electrode and terminal 208.

The left ventricular lead system includes conductors 110, 112 for transmitting sense and pacing signals between terminals 210, 212 of the hybrid device and LV-tip and LV-ring electrodes respectively. The left atrial lead system includes conductor 114 for transmitting signals between the LA-tip electrode and terminal 214 and conductor 116 for transmitting signals between the LA-ring electrode and terminal 216. A can electrode 209 coupled to a housing 130 of the hybrid device is also provided.

The device circuitry 203 is encased in a hermetically sealed housing 130 suitable for implanting in a human body. Power to the hybrid device 200 is supplied by an energy source 233, such as an electrochemical battery, fuel cell, or external energy source, that is housed within, or otherwise supplies energy to, the device 200. In one embodiment, the hybrid circuitry 203 is a programmable microprocessor-based system, including a control system 220, detector system 230, pacemaker 240, cardioverter/defibrillator pulse generator 250 and a memory circuit 261. The memory circuit 261 stores parameters for various pacing, defibrillation, and sensing modes and stores data indicative of cardiac signals received by other components of the device circuitry 203. A memory is also provided for storing historical EGM and therapy data 262, which may be used on-board for various purposes and transmitted to an external programmer unit 280 as required.

The control system 220 may use various control subsystems including pacemaker control 221, cardioverter/defibrillator control 224, and arrhythmia detector 222. The control system 220 may encompass additional functional components (not shown) for controlling the device circuitry 203. The control system 220 and memory circuit 261 cooperate with other components of the device circuitry 203 to perform operations involving synchronized pacing, in addition to other sensing, pacing and defibrillation functions.

Telemetry circuitry 270 is additionally coupled to the device circuitry 203 to allow the hybrid device 200 to communicate with an external programmer unit 280. In one embodiment, the telemetry circuitry 270 and the programmer unit 280 use a wire loop antenna and a radio frequency telemetric link to receive and transmit signals and data between the programmer unit 280 telemetry circuitry 270. In this manner, programming commands may be transferred to the device circuitry 203 from the programmer unit 280 during and after implant. In addition, stored cardiac data relevant to synchronized pacing therapy, along with other data, may be transferred to the programmer unit 280 from the hybrid device 200, for example.

Cardiac signals sensed through use of the RV-tip and LV-tip electrodes are near-field signals as are known in the art. More particularly, a signal derived from the right ventricle is detected as a voltage developed between the RV-tip electrode and the RV-coil. RV-tip and RV-coil electrodes are shown coupled to an RV-sense amplifier 231 located within the detector system 230. Signals received by the RV-sense amplifier 231 are communicated to the signal processor and A/D converter 239. The RV-sense amplifier 231 serves to sense and amplify the signals. The signal processor and A/D converter 239 convert the R-wave signals from analog to digital form and communicate the signals to the control system 220. Signals derived from the left ventricle are detected as a voltage developed between the LV-tip electrode and the LV-ring electrode. LV-tip and LV-ring electrodes are shown coupled to an LV-sense amplifier 233 located within the detector system 230. Signals received by the 233 are communicated to the signal processor and A/D converter 239. The LV-sense amplifier 233 serves to sense and amplify the signals. The signal processor and A/D converter 239 convert the R-wave signals from analog to digital form and communicate the signals to the control system 220.

Cardiac signals sensed through use of one or both of the RV-coil and the SVC-coil are far-field signals, also referred to as morphology or shock channel signals, as are known in the art. More particularly, a shock channel signal is detected as a voltage developed between the RV-coil and the SVC-coil. A shock channel signal may also be detected as a voltage developed between the RV-coil and the SVC-coil coupled to the can electrode 209. Shock channel signals developed using appropriate combinations of the RV-coil, SVC-coil, and can electrode are sensed and amplified by a shock EGM amplifier 236 located in the detector system 230. The output of the EGM amplifier 236 is coupled to the control system 220 via the signal processor and A/D converter 239.

RA-tip and RA-ring electrodes are shown coupled to an RA-sense amplifier 232 located within the detector system 230. Atrial sense signals received by the RA-sense amplifier 232 in the detector system 230 are communicated to an A/D converter 239. The RA-sense amplifier serves to sense and amplify the A-wave signals of the right atrium. The A/D converter 239 converts the sensed signals from analog to digital form and communicates the signals to the control system 220.

A-wave signals originating in the left atrium are sensed by the LA-tip and LA-ring electrodes. The A-waves are sensed and amplified by the LA-sense amplifier 234 located in the detector system. The LA-sense amplifier serves to sense and amplify the A-wave signals of the left atrium. The A/D converter 239 converts the sensed signals from analog to digital form and communicates the signals to the control system 220.

The pacemaker 240 communicates pacing signals to the pacing electrodes, RV-tip, RA-tip, LV-tip and LA-tip, according to a pre-established pacing regimen under appropriate conditions. Blanking circuitry (not shown) is employed in a known manner when ventricular or atrial pacing pulses are delivered, such that the ventricular channels, atrial channels, and shock channel are properly blanked at the appropriate time and for the appropriate duration.

A hybrid device that incorporates CFM functionality may be configured to improve pumping function by altering contraction sequences in a manner distinct from conventional bradycardia pacing. To treat bradycardia, for example, pacing may be performed when the heart rate is not fast enough or the atrioventricular (AV) interval is too long. Thus, patients with intact AV conduction and adequate ventricular rates may not be paced at all if, following a sensed intrinsic atrial event, AS, AV conduction occurs before the programmed AV interval has elapsed and an intrinsic ventricular event, VS, is sensed.

To improve pumping function, two or more heart chambers may be paced simultaneously or in phased sequence, thus coordinating inefficient or non-existent contraction sequences. For example, a pacing mode may be employed to pace both the left ventricle, LVP, and the right ventricle, RVP, after a sensed atrial contraction, AS. Such a pacing mode may mitigate pathological ventricular conduction delays, thereby improving the pumping function of the heart.

FIGS. 6 and 7 illustrate various components of the transthoracic system of a hybrid transthoracic/intrathoracic cardiac stimulation device according to an embodiment of the present invention. According to the configuration shown in FIG. 6, a hybrid device incorporates a processor-based control system 305 which includes a micro-processor 306 coupled to appropriate memory (volatile and non-volatile) 309, it being understood that any logic-based control architecture can be used. The control system 305 is coupled to circuitry and components to sense, detect, and analyze electrical signals produced by the heart and deliver electrical stimulation energy to the heart under predetermined conditions to treat cardiac arrhythmias. In certain configurations, the control system 305 and associated components can also provide pacing therapy to the heart. The electrical energy delivered by the hybrid device may be in the form of low energy pacing pulses or high energy pulses for cardioversion or defibrillation.

Cardiac signals are sensed using the subcutaneous electrode(s) 314 and the can or indifferent electrode 307 provided on the hybrid device housing. Cardiac signals can also be sensed using only the subcutaneous electrodes 314, such as in a non-active can configuration. As such, unipolar, bipolar, or combined unipolar/bipolar electrode configurations may be employed. The sensed cardiac signals are received by sensing circuitry 304, which includes sense amplification circuitry and may also include filtering circuitry and an analog-to-digital (A/D) converter. The sensed cardiac signals processed by the sensing circuitry 304 may be received by noise reduction circuitry 303, which can further reduce noise before signals are sent to the detection circuitry 302. Noise reduction circuitry 303 may also be incorporated after detection circuitry 302 in cases where high power or computationally intensive noise reduction algorithms are required.

In the illustrative configuration shown in FIG. 6, the detection circuitry 302 is coupled to, or otherwise incorporates, noise reduction circuitry 303. The noise reduction circuitry 303 operates to improve the signal-to-noise ratio of sensed cardiac signals by removing noise content of the sensed cardiac signals introduced from various sources. Typical types of transthoracic cardiac signal noise includes electrical noise and noise produced from skeletal muscles, for example. A number of methodologies for improving the signal-to-noise ratio of sensed cardiac signals in the presence of skeletal muscular induced noise, including signal separation techniques, are described in further detail in the above-identified provisional application.

According to another aspect, skeletal muscular noise can be used as a useful artifact signal for a variety of purposes. In one approach, the detection circuitry 302 and noise reduction circuitry 303 cooperate to detect skeletal muscular noise, and the detected skeletal muscular noise can be used to determine the activity level of the patient. The activity level information derived from the detected skeletal muscular noise can be used for a number of purposes, such as minimizing the delivery of inappropriate cardioversion and defibrillation therapy, as is described in further detail in the above-identified provisional application.

Detection circuitry 302 typically includes a signal processor that coordinates analysis of the sensed cardiac signals and/or other sensor inputs to detect cardiac arrhythmias, such as, in particular, tachyarrhythmia. Rate-based (e.g., rate zone-based), pattern and rate-based, and/or morphological discrimination algorithms can be implemented by the signal processor of the detection circuitry 302 to detect and verify the presence and severity of an arrhythmic episode.

Exemplary arrhythmia detection and discrimination circuitry, structures, and techniques, aspects of which can be implemented by a hybrid device of a type contemplated herein, are disclosed in commonly owned U.S. Pat. Nos. 5,301,677 and 6,438,410, which are hereby incorporated herein by reference in their respective entireties. Exemplary pattern and rate-based arrhythmia detection and discrimination circuitry, structures, and techniques, aspects of which can be implemented by a hybrid device of a type contemplated herein, are disclosed in U.S. Pat. Nos. 6,487,443; 6,259,947; 6,141,581; 5,855,593; and 5,545,186, which are hereby incorporated herein by reference in their respective entireties. Arrhythmia detection methodologies particularly well suited for implementation in subcutaneous cardiac stimulation systems are described in further detail in the above-identified provisional application.

The detection circuitry 302 communicates cardiac signal information to the control system 305. Memory circuitry 309 of the control system 305 contains parameters for operating in various sensing, defibrillation, and pacing modes, and stores data indicative of cardiac signals received by the detection circuitry 302. The memory circuitry 309 can also be configured to store historical ECG and therapy data, which may be used for various purposes and transmitted to an external receiving device as needed or desired.

In certain configurations, the hybrid device can include diagnostics circuitry 310. The diagnostics circuitry 310 typically receives input signals from the detection circuitry 302 and the sensing circuitry 304. The diagnostics circuitry 310 provides diagnostics data to the control system 305, it being understood that the control system 305 can incorporate all or part of the diagnostics circuitry 310 or its functionality. The control system 305 may store and use information provided by the diagnostics circuitry 310 for a variety of diagnostics purposes. This diagnostic information may be stored, for example, subsequent to a triggering event or at predetermined intervals, and may include system diagnostics, such as power source status, therapy delivery history, and/or patient diagnostics. The diagnostic information may take the form of electrical signals or other sensor data acquired immediately prior to and after therapy delivery.

According to a configuration that provides transthoracic cardioversion and defibrillation therapies, the control system 305 processes cardiac signal data received from the detection circuitry 302 and initiates appropriate tachyarrhythmia therapies to terminate cardiac arrhythmic episodes and return the heart to normal sinus rhythm. The control system 305 is coupled to shock therapy circuitry 316. The shock therapy circuitry 316 is coupled to the subcutaneous electrode(s) 314 and the can or indifferent electrode 307 of the hybrid device housing. Upon command, the shock therapy circuitry 316 delivers cardioversion and defibrillation stimulation energy to the heart in accordance with a selected cardioversion or defibrillation therapy. In a less sophisticated configuration, the shock therapy circuitry 316 is controlled to deliver defibrillation therapies, in contrast to a configuration that provides for delivery of both cardioversion and defibrillation therapies. Exemplary ICD high energy delivery circuitry, structures and functionality, aspects of which can be incorporated in a hybrid device of a type contemplated herein, are disclosed in commonly owned U.S. Pat. Nos. 5,372,606; 5,411,525; 5,468,254; and 5,634,938, which are hereby incorporated herein by reference in their respective entireties.

In accordance with another configuration, the transthoracic system of a hybrid device can incorporate a cardiac pacing capability in addition to cardioversion and/or defibrillation capabilities. As is shown in dotted lines in FIG. 6, the hybrid device can include pacing therapy circuitry 330 which is coupled to the control system 305 and the subcutaneous and can/indifferent electrodes 314, 307. Upon command, the pacing therapy circuitry delivers pacing pulses to the heart in accordance with a selected pacing therapy. Control signals, developed in accordance with a pacing regimen by pacemaker circuitry within the control system 305, are initiated and transmitted to the pacing therapy circuitry 330 where pacing pulses are generated. A pacing regimen may be modified by the control system 305.

A number of cardiac pacing therapies can be delivered via the pacing therapy circuitry 330 as shown in FIG. 6. Alternatively, cardiac pacing therapies can be delivered via the shock therapy circuitry 316, which effectively obviates the need for separate pacemaker circuitry. Examples of various approaches for delivering cardiac pacing therapies via the shock therapy circuitry 316 are disclosed in commonly owned U.S. patent application Ser. No. 10/377,274 (Attorney Docket No. GUID.602PA), filed Feb. 28, 2003, which is hereby incorporated herein by reference.

The hybrid device shown in FIG. 6 can be configured to receive signals from one or more physiologic and/or non-physiologic sensors 312. Depending on the type of sensor employed, signals generated by the sensors 312 can be communicated to transducer circuitry coupled directly to the detection circuitry or indirectly via the sensing circuitry. It is noted that certain sensors can transmit sense data to the control system 305 without processing by the detection circuitry 302.

Communications circuitry 318 is coupled to the micro-processor 306 of the control system 305. The communications circuitry 318 allows the hybrid device to communicate with one or more receiving devices or systems situated external to the hybrid device. By way of example, the hybrid device can communicate with a patient-worn, portable or bed-side communication system via the communications circuitry 318. In one configuration, one or more physiologic or non-physiologic sensors (subcutaneous, cutaneous, or external of patient) can be equipped with a short-range wireless communication interface, such as an interface conforming to a known communications standard, such as Bluetooth or IEEE 802 standards. Data acquired by such sensors can be communicated to the hybrid device via the communications circuitry 318. It is noted that physiologic or non-physiologic sensors equipped with wireless transmitters or transceivers can communicate with a receiving system external of the patient.

The communications circuitry 318 can allow the hybrid device to communicate with an external programmer. In one configuration, the communications circuitry 318 and the programmer unit (not shown) use a wire loop antenna and a radio frequency telemetric link, as is known in the art, to receive and transmit signals and data between the programmer unit and communications circuitry 318. In a manner similar to that described above with regard to the intrathoracic system block diagram of FIG. 5, programming commands and data can be transferred between the hybrid device and the programmer unit during and after implant. Using a programmer, a physician is able to set or modify various parameters used by the hybrid device. For example, a physician can set or modify parameters affecting sensing, detection, pacing, and defibrillation functions of the hybrid device, including pacing and cardioversion/defibrillation therapy modes.

Power to the hybrid device is supplied by a power source 320 disposed within a hermetically sealed housing of the hybrid device. The power source 320 can be the same (or a different) source of power as the power source 233 shown in FIG. 5. In one configuration, the power source 320 includes a rechargeable battery. According to this configuration, charging circuitry is coupled to the power source 320 to facilitate repeated non-invasive charging of the power source 320. The communications circuitry 318, or separate receiver circuitry, is configured to receive RF energy transmitted by an external RF energy transmitter. The hybrid device may, in addition to a rechargeable power source, include a non-rechargeable battery. It is understood that a rechargeable power source need not be used, in which case a long-life non-rechargeable battery is employed.

FIG. 7 illustrates a configuration of detection circuitry 402 of the transthoracic system of a hybrid device, which includes one or both of rate detection circuitry 410 and morphological analysis circuitry 412. Detection and verification of arrhythmias can be accomplished using rate-based discrimination algorithms as known in the art implemented by the rate detection circuitry 410. Arrhythmic episodes can also be detected and verified by morphology-based analysis of sensed cardiac signals as is known in the art. Tiered or parallel arrhythmia discrimination algorithms can also be implemented using both rate-based and morphologic-based approaches.

A hybrid device of the present invention can be configured to provide enhanced rhythm analysis and discrimination. According to one hybrid device configuration, an intrathoracic lead system can include an atrial lead having one or more atrial electrodes. A controller of the hybrid device can configure the device to operate in a mode that facilitates tachyarrhythmia discrimination using one or more subcutaneous electrodes and one or more atrial electrodes. For example, the controller can discriminate tachyarrhythmias having a ventricular origin from tachyarrhythmias having an atrial origin.

By way of further example, a hybrid device can be configured to provide subcutaneous and epicardial sensing to verify cardiac rhythms and to improve discrimination of rhythms, such as by discriminating atrial fibrillation from noise. According to another configuration, a transvenous-based ventricular system without an atrial lead can cooperate with a subcutaneous lead for improving discrimination of ventricular and atrial arrhythmias.

The detection circuitry 402, which is coupled to a micro-processor 406, can be configured to incorporate, or communicate with, specialized circuitry for processing sensed cardiac signals in manners particularly useful in a transthoracic cardiac stimulation device. As is shown by way of example in FIG. 7, the detection circuitry 402 can receive information from multiple physiologic and non-physiologic sensors. As illustrated, transthoracic acoustics can be monitored using an appropriate acoustic sensor. Heart sounds, for example, can be detected and processed by cardiac acoustic processing circuitry 418 for a variety of purposes. The acoustics data is transmitted to the detection circuitry 402, via a hardwire or wireless link, and used to enhance cardiac signal detection. For example, acoustics can be used to discriminate normal cardiac sinus rhythm with electrical noise from potentially lethal arrhythmias, such as ventricular tachycardia or ventricular fibrillation.

The detection circuitry 402 can also receive information from one or more sensors that monitor skeletal muscle activity. In addition to cardiac activity signals, skeletal muscle signals are readily detected by transthoracic electrodes. Such skeletal muscle signals can be used to determine the activity level of the patient. In the context of cardiac signal detection, such skeletal muscle signals are considered artifacts of the cardiac activity signal, which can be viewed as noise. Processing circuitry 416 receives signals from one or more skeletal muscle sensors, and transmits processed skeletal muscle signal data to the detection circuitry 402. This data can be used to discriminate normal cardiac sinus rhythm with skeletal muscle noise from cardiac arrhythmias.

As was previously discussed, the detection circuitry 402 is preferably coupled to, or otherwise incorporates, noise processing circuitry 414. The noise processing circuitry 414 processes sensed cardiac signals to improve the signal-to-noise ratio of sensed cardiac signals by removing or rejecting noise content of the sensed cardiac signals.

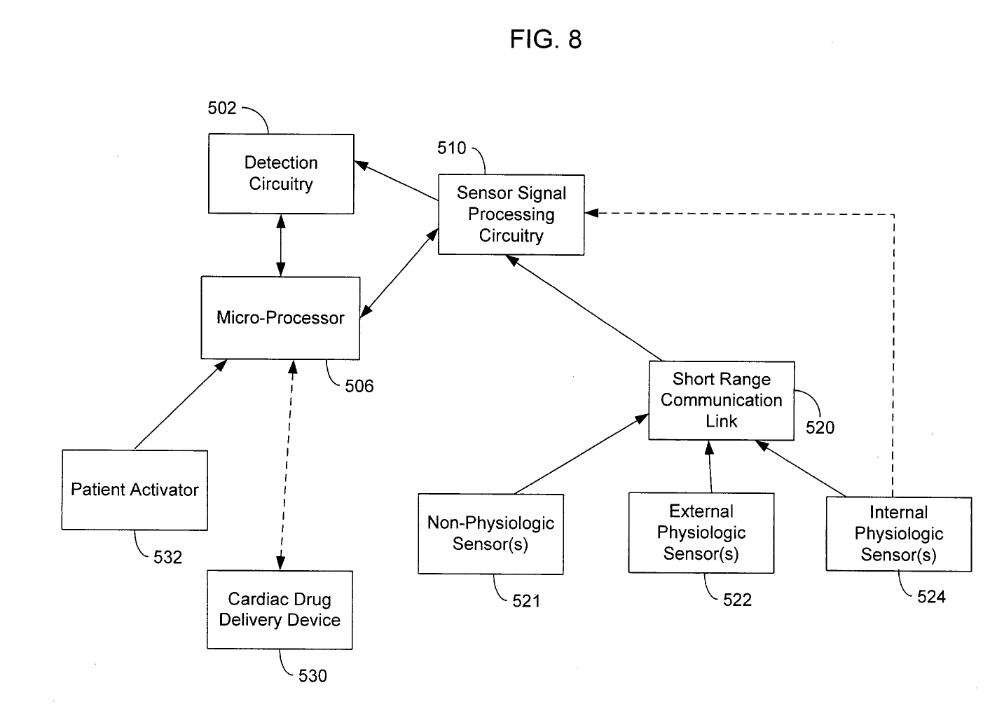

Turning now to FIG. 8, there is illustrated a block diagram of various components that can be incorporated into embodiments of a hybrid device in accordance with the present invention. FIG. 8 shows a number of components that are associated with detection of various physiologic and non-physiologic parameters. As shown, the hybrid device includes a micro-processor 506, which is typically incorporated in a control system for the hybrid device, coupled to detection circuitry 502. Sensor signal processing circuitry 510 can receive sensor data from a number of different sensors.

For example, a hybrid device can cooperate with, or otherwise incorporate, various types of non-physiologic sensors 521, external or cutaneous physiologic sensors 522, and/or internal physiologic sensors 524. Such sensors can include an acoustic sensor, an impedance sensor, an oxygen saturation sensor, and a blood pressure sensor, for example. Each of these sensors 521, 522, 524 can be communicatively coupled to the sensor signal processing circuitry 510 via a short range wireless communication link 520. Certain sensors, such as an internal physiologic sensor 524, can alternatively be communicatively coupled to the sensor signal processing circuitry 510 via a wired connection (e.g., electrical or optical connection).

A cardiac drug delivery device 530 can be employed to cooperate with a hybrid device of a type contemplated herein. For example, the cardiac drug delivery device 530 can deliver one or more anti-arrhythmic agents that have been approved for the chemical treatment of tachycardia and fibrillation. A non-exhaustive, non-limiting list of such agents includes: quinidine, procainamide, disopyramide, flecaininde, propafenone, moricizine, sotalol, amiodarone, ibutilide, and dofetilide (e.g., class I and III anti-arrhythmic agents). These and other drugs can be delivered prior to, during, and after delivery of cardioversion/defibrillation therapy for purposes of enhancing patient comfort, lowering defibrillation thresholds, and/or chemically treating an arrhythmic condition.

In accordance with another configuration, the hybrid device can include a non-implanted patient actuatable activator 532 that operates in cooperation with the hybrid device. The activator 532 includes a communication unit and produces an activation signal in response to a patient sensing a perceived severe arrhythmic condition. Alternatively, or in addition, the activation signal may be produced by the non-implanted activator 532 in response to the hybrid device detecting the arrhythmic condition. The hybrid device includes communication circuitry for communicating with the non-implanted activator 532.

The activator 532 can be actuated by the patient or person attending the patient to initiate cardioversion/defibrillation therapy. Typically, the hybrid device, in response to receiving an activation signal, confirms that the patient is experiencing an actual adverse cardiac condition prior to initiating appropriate therapy. The non-implanted activator 532, in communication with the hybrid device, can also generate a patient perceivable initiating signal to indicate manual or automatic commencement of a drug delivery regimen to treat the actual adverse cardiac condition.

The activator 532 can be configured to include an inhibit button that allows the patient to override the delivery of a stimulation therapy in the event that the hybrid device indicates that a potentially serious arrhythmia has been detected, but the patient determines that the detection indication is in error. Unambiguous arrhythmic episodes detected by the hybrid device are preferably subject to therapy delivery upon detection and confirmation, notwithstanding receipt of an inhibition signal from the patient activator 532.

The components, functionality, and structural configurations depicted in FIGS. 1-8 are intended to provide an understanding of various features and combination of features that can be incorporated in a hybrid device of the present invention. It is understood that a wide variety of hybrid device configurations are contemplated, ranging from relatively sophisticated to relatively simple designs. As such, particular hybrid device configurations can include particular features as described herein, while other such device configurations can exclude particular features described herein.

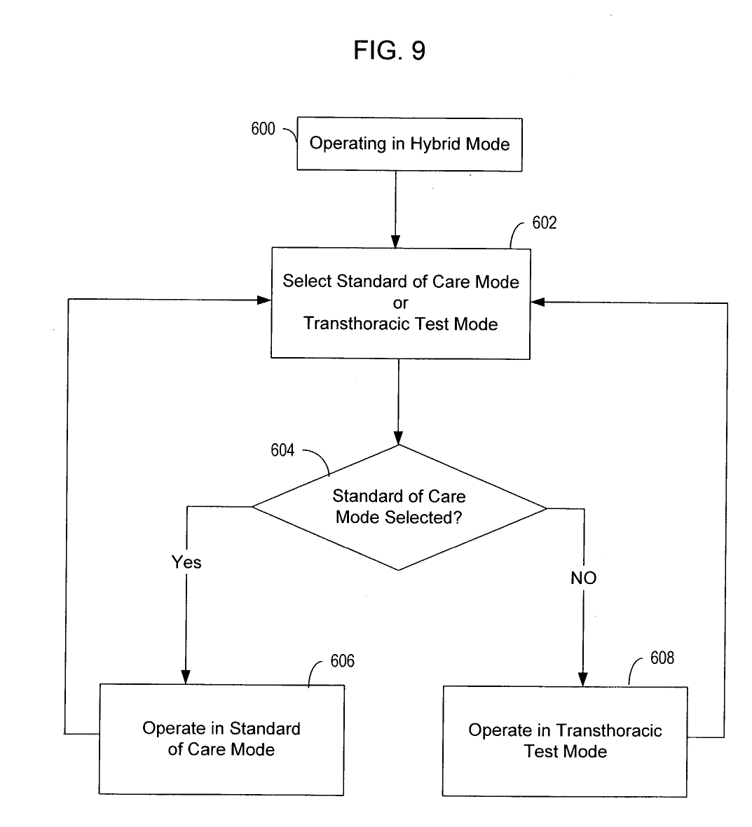

FIGS. 9-18 illustrate several methodologies that can be implemented using a hybrid transthoracic/intrathoracic cardiac stimulation device of the present invention. The methodologies described with reference to FIGS. 9-18 are intended to represent a non-exhaustive, non-limiting recitation of various useful methodologies that can be implemented using a hybrid device of the present invention.

FIG. 9 illustrates several processes involving basic mode switching during hybrid device operation. During operation 600, the hybrid device can be configured 602 to operate in a standard of care mode or a tranthoracic test mode. Selection of a particular hybrid device operating mode can be effected in several ways, such as in response to an externally initiated command (e.g., via a programmer) or in response to software instructions. If a standard of care mode is selected 604, the hybrid device performs a mode switch so that operation in the standard of care mode commences 606. If a transthoracic test mode is selected 604, the hybrid device performs a mode switch so that operation in the transthoracic test mode commences 608. Subsequent hybrid device operating mode selections can be made at block 602.

In FIG. 9, processes involving hybrid device mode switching between two modes or system configurations are depicted. It is understood that more than two operating modes or system configurations can be selected for operation in hybrid devices that provide such additional operating modes. Moreover, as will be described below, the operating modes of a hybrid device can be selected such that only one of the selectable modes is operative at any given time or, alternatively, multiple modes can be selected for concurrent or combinational operation.

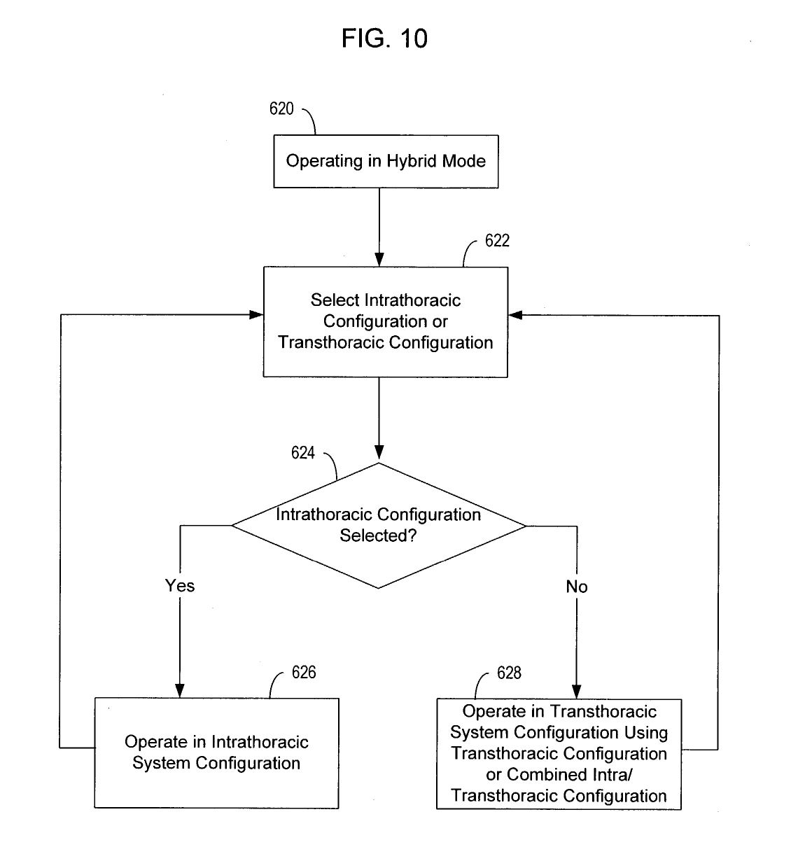

FIG. 10 illustrates several processes involving mode switching during hybrid device operation in accordance with one embodiment. According to this embodiment, a hybrid device is selectively configurable to operate in an intrathoracic configuration, a transthoracic configuration, or a combined intrathoracic/transthoracic configuration. During operation 620, the hybrid device can be configured 622 to operate in an intrathoracic configuration or a transthoracic configuration. Selecting the operating configuration of the hybrid device can be effected in several ways, such as those discussed above with regard to FIG. 9.

If an intrathoracic configuration is selected 624, the hybrid device configures its circuitry for operation 626 in an intrathoracic system configuration. Alternatively, the hybrid device can configure its circuitry for operation 628 in a transthoracic system configuration, which can implicate a transthoracic-only configuration or a combined intrathoracic/transthoracic configuration.

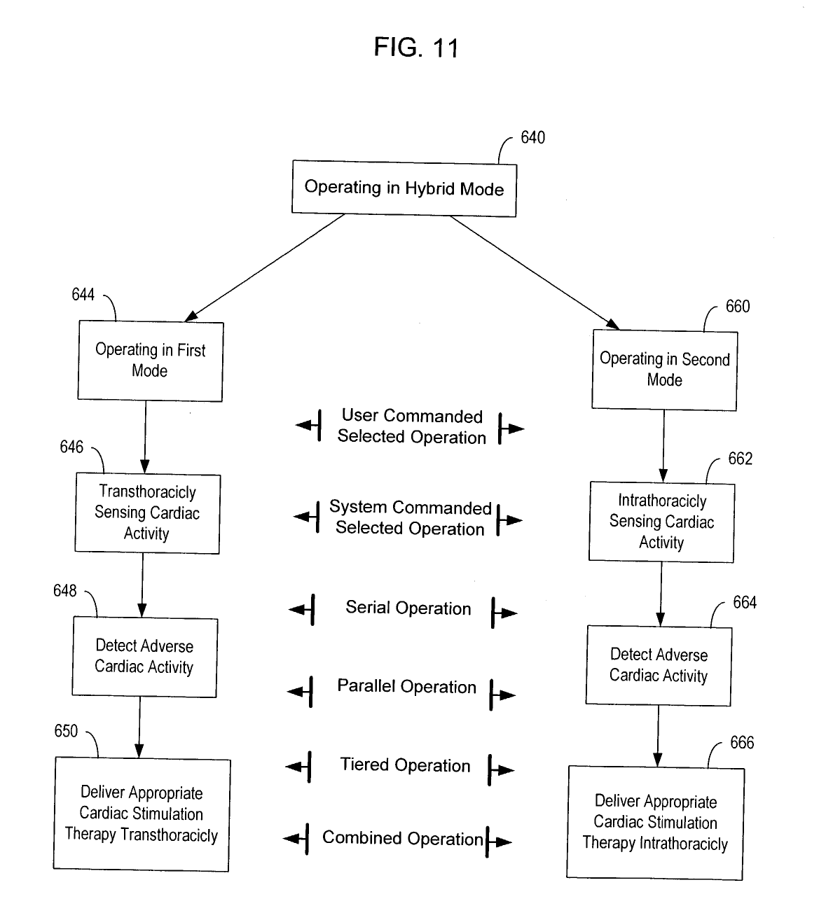

FIG. 11 illustrates various ways in which functions associated with two or more hybrid device operating modes can be selected for operation. It is assumed for purposes of explanation that the subject hybrid device is operable 640 in at least a first mode 644 and a second mode 660. Operating in the first mode 644, according to this illustrative embodiment, involves transthoracicly sensing 646 cardiac activity and, in response to detecting 648 adverse cardiac activity (e.g., tachycardia or bradycardia), transthoracicly delivering 650 appropriate cardiac stimulation therapy. Operating in the second mode 660 involves intrathoracicly sensing 662 cardiac activity and, in response to detecting 664 adverse cardiac activity, intrathoracicly delivering 666 appropriate cardiac stimulation therapy.

As is further shown in FIG. 11, the first and second modes can be selected for operation in a variety of ways. Also, the manner in which the first and second modes operate relative to one another can be selected in a variety of ways. Further, individual functions or groups of functions associated with the first and second modes can be selectively implemented in a variety of ways. Various ways of effecting operating mode selectivity are depicted in FIG. 11, as denoted by the central text provided between left and right arrows in FIG. 11. Each of the operating mode selection options shown in the central text can be implemented for individual or multiple hybrid device functions.

For example, the first and second modes 644, 660, and functions associated therewith (e.g., 646-650 and 662-666, respectively), can be selected for operation in response to a user command, such as a command initiated by a clinician through use of a programmer or other external command device. The first and second modes, and functions associated therewith, can also be selected for operation in response to hybrid device commands or program instructions. The first and second modes, and functions associated therewith, can further be selected for serial operation, parallel operation, tiered operation, or combined operation.

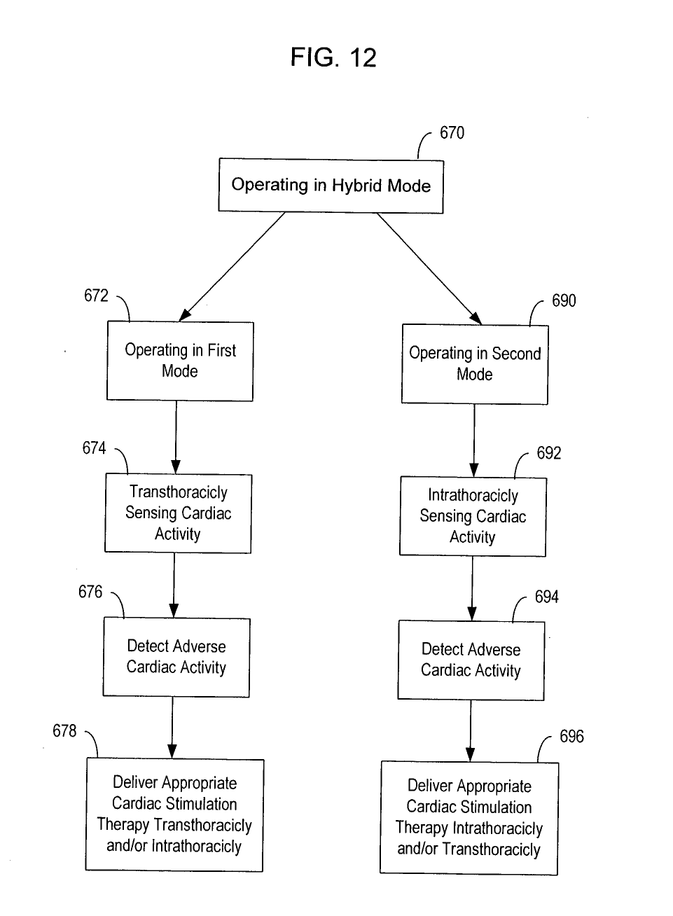

FIG. 12 illustrates an embodiment of a hybrid device in which two modes and their associated functions can be selected to perform various operations in accordance with a desired sequence. In this particular embodiment, the hybrid device can operate 670 in a first mode 672 or in a second mode 690. The first mode, in this illustrative example, implements a hybrid device configuration that provides for transthoracic sensing 674 of cardiac activity. The second mode implements a hybrid device configuration that provides for intrathoracic sensing 692 of cardiac activity. In each of the modes, the hybrid device is configured to detect 676, 694 adverse cardiac activity. In response to same, the hybrid device can be configured to deliver 678, 696 appropriate cardiac stimulation therapy transthoracicly and/or intrathoracicly.

It is appreciated that many other combinations of modes and functions associated with intrathoracic and transthoracic system operation can be selectively implemented, and those combinations described herein are provided as illustrative examples of such possible combinations. The following are additional non-limiting examples that illustrate several scenarios in which a hybrid device can find particular usefulness:

### EXAMPLE #1

### Simultaneous Mode

A patient may have a history of monomorphic ventricular tachyarrhythmia (MVT) progressing to polymorphic ventricular tachyarrhythmia (PVT) and then to ventricular fibrillation (VF) (i.e., MVT→PVT→VF), and have high defibrillation thresholds at implant (e.g., does not have adequate safety margin with a conventional 31 J transvenous-based system). Such a patient may be a candidate for a hybrid device implemented in the following manner. The intrathoracic system of the hybrid device can be programmed to discriminate tachycardia from VF, and to apply antitachycardia pacing and/or cardioversion during MVT. The transthoracic system can be enabled to detect VF and apply defibrillation therapy.

### EXAMPLE #2

### Tiered Mode

A hybrid device can be implanted in test patient population. A test system (e.g., transthoracic system employing subcutaneous electrode configuration only) of the hybrid device can be programmed to operate first, with a standard of care system (e.g., conventional intrathoracic system) being dormant in terms of therapy delivery. If the test system fails to convert an arrhythmia after x attempts, the hybrid device switches operation to the standard of care system. If the standard of care system fails to convert an arrhythmia after y attempts, the hybrid device combines circuitry and/or functionality of both systems to define a new system configuration and attempts to convert the arrhythmia using both systems.

### EXAMPLE #3

### Tiered Mode

A hybrid device can be implanted in test patient population. A standard of care system (e.g., conventional intrathoracic system) of the hybrid device can be programmed to operate first, with a test system (e.g., transthoracic system employing subcutaneous electrode configuration only) being dormant in terms of therapy delivery. If the standard of care system fails to convert an arrhythmia after x attempts, the hybrid device switches operation to the test system. The hybrid device then attempts to convert the arrhythmia using the test system. In an alternate approach, if lead integrity is compromised, the test system can be used as backup to the standard of care system to reduce the urgency for a clinic visit.

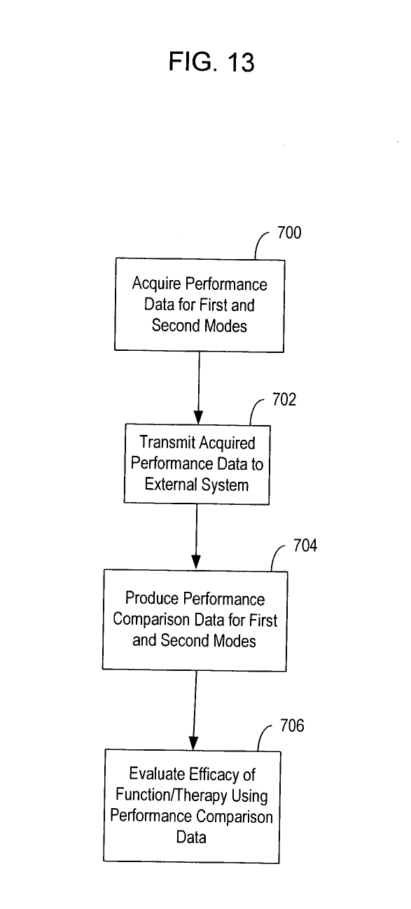

As was discussed previously, a hybrid device can be particularly useful in providing a direct comparison between new system/function performance verses established system/function performance. FIG. 13 depicts one such system configuration in which performance data is acquired 700 by the hybrid device and/or an external monitoring device while the hybrid device operates in a first mode and a second mode. This data can be acquired and stored within the hybrid device for later transmission 702 to an external system. Alternatively, the performance data can be acquired in real-time and transmitted in real-time to an external system. It is noted that the external system can be situated local to the patient, as in the case of a programmer, or distant from the patient, such as a system communicatively coupled to a programmer or other interrogation device via a communication link (e.g., network connection).

The external system processes 704 the received performance data and produces various forms of comparison data that facilitate evaluation of hybrid device performance when operating in the first mode in comparison to the second mode (or vice versa). Using the comparison data, the efficacy of a particular function or therapy can be evaluated 706 using computer assisted and/or manual means.

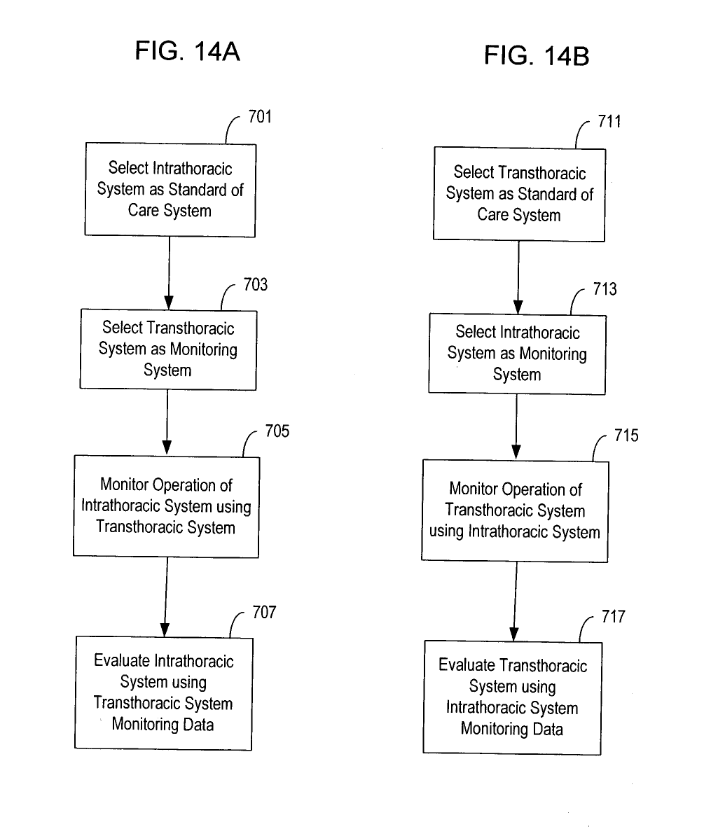

A hybrid device of the present invention can provide other system/function evaluation opportunities heretofore unavailable using conventional approaches. As is shown in FIG. 14A, the intrathoracic system of a hybrid device can be selected 701 as a standard of care system. The transthoracic system of a hybrid device can be selected 703 as a monitoring system. In this system configuration, the hybrid device monitors 705 operation of the intrathoracic system using the transthoracic system. It is noted that the hybrid device can also monitor operation of the intrathoracic system using the intrathoracic system itself, but that the transthoracic system can acquire monitoring data different from that obtainable using only the intrathoracic system. Performance of the intrathoracic system can be evaluated 707 using the monitoring data acquired by the transthoracic system or the combined systems.

FIG. 14B illustrates another evaluation/monitoring scenario by which the transthoracic system is selected 711 as a standard of care system, and the intrathoracic system can be selected 713 as a monitoring system. In this system configuration, the hybrid device monitors 715 operation of the transthoracic system using the intrathoracic system, it being understood that the hybrid device can also monitor operation of the transthoracic system using the transthoracic system itself, and that the intrathoracic system can acquire monitoring data different from that obtainable using only the transthoracic system. Performance of the transthoracic system can be evaluated 717 using the monitoring data acquired by the intrathoracic system or the combined systems.

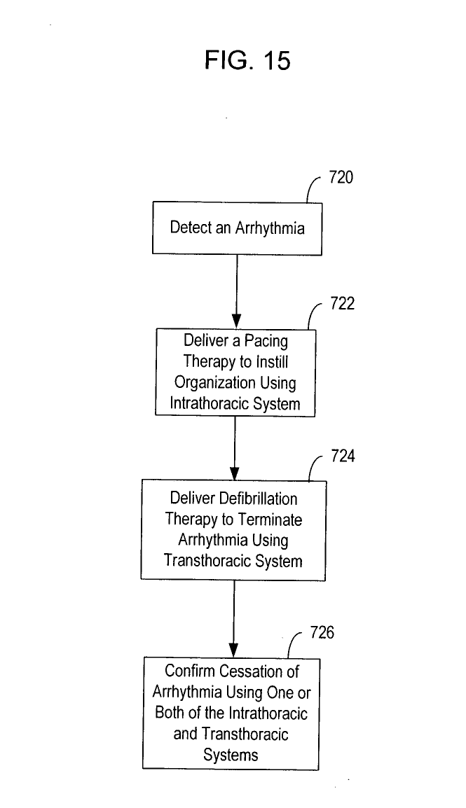

FIG. 15 illustrates another capability that is realizable through employment of a hybrid device of the present invention. A hybrid device can advantageously perform a particular function in one mode or configuration which enhances performance of a second function performed in another mode or configuration. For example, the controller of a hybrid device can configure the device to operate in a first configuration (e.g., intrathoracic configuration) to perform a first function. The controller can then configure the hybrid device to operate in a second configuration (e.g., transthoracic configuration) to perform a second function, such that performance of the first function enhances performance of the second function.

In accordance with the specific example illustrated in FIG. 15, a hybrid device can be implemented to deliver combinations of therapies to treat various types of arrhythmias. FIG. 15 depicts one such approach for treating an arrhythmia using a combination of pacing and defibrillation therapies delivered by the respective intrathoracic and transthoracic systems of a hybrid device.

After detecting 720 an arrhythmia, and after confirming an arrhythmic episode, the hybrid device can deliver 722 a pacing therapy using the intrathoracic system to instill organization in the cardiac rhythms. Assuming that the pacing therapy fails to convert the arrhythmia to normal sinus rhythm, the hybrid device delivers 724 a defibrillation therapy using the transthoracic system to terminate the arrhythmia. The hybrid device confirms 726 the cessation or persistence of the arrhythmia using one or one or both of the intrathoracic and transthoracic systems. If the arrhythmia persists, additional therapies can be delivered by the hybrid device using one or one or both of the intrathoracic and transthoracic systems in an attempt to terminate the arrhythmia.

According to the methodology illustrated in FIG. 15, a hybrid device can be configured to deliver a single electrical therapy applied to a selected region of selected cardiac tissue, wherein the single electrical therapy comprises the combination of multiple therapies. One specific implementation of the methodology depicted in FIG. 15 involves delivery of two discrete therapies: a pacing level therapy applied to a localized portion of a region of the selected cardiac tissue having relatively low susceptibility to defibrillation-level shock field strengths followed by, or occurring simultaneously with, a defibrillation therapy applied to portions of the tissue having regions of fibrillating myocardium over which the sub-defibrillation level shocks exert control. Such regions of fibrillating myocardium are preferably those characterized by a 1:1 phase lock of a local electrogram of any region to a stimulus artifact of that region.

The selected cardiac tissue may be ventricular tissue, or it may be atrial tissue. In the case of atrial tissue, the first defibrillation shock which would otherwise occur within the vulnerable period (T-wave) of the ventricular activation cycle, should not occur until after ventricular depolarization. The first defibrillation-level shock preferably occurs coincident with or after the last pacing level shock. For example, the last pacing level shock preferably occurs not sooner than the beginning of an optimum period beginning before the first defibrillation-level shock. This period can be determined by extracting a feature from sensed cardiac signals, such as morphology of the ECG or some component of the ECG; some fraction (e.g., 80-100%) of the cardiac cycle length, etc. The exact condition used to determine the optimum period is typically determined empirically by the particular clinical and therapeutic context; however, typical practical limits on the optimum period would be from 250 milliseconds prior to the first defibrillation shock, to coincident (or simultaneously), i.e., within less than one millisecond, with the first defibrillation shock.

The combined therapy delivery approach depicted in FIG. 15 effectively reduces the voltage and/or energy required for successful defibrillation by the first defibrillation-level shock. While the region controlled by the pacing level shocks may be only the same size as the localized region, the objective of this procedure is for the successive regions of fibrillating myocardium to be successively larger in terms of the amount of tissue controlled. A successively larger amount of controlled tissue increases the probability that the entire heart may be successfully treated by a single defibrillation shock, and especially so by a single defibrillation shock of reduced strength than would otherwise be possible. Additional details of combined pacing/defibrillation therapies implemented by a conventional device but adaptable for use in a hybrid device of the present invention are disclosed in commonly owned U.S. Pat. No. 5,797,967, which is hereby incorporated herein by reference.

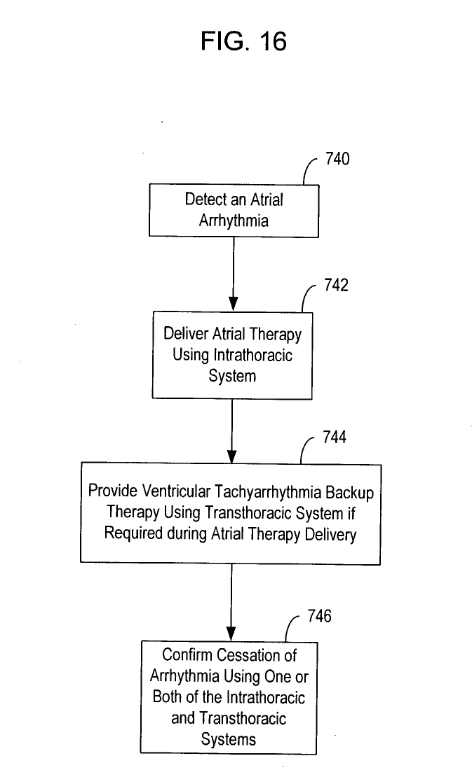

FIG. 16 illustrates another capability which can be realized using a hybrid device which employs both transthoracic and intrathoracic systems operating in cooperation. According to the methodology depicted in FIG. 16, a hybrid device can be configured to provide various therapies to the atria while providing added safety features to prevent ventricular arrhythmia. As shown in FIG. 16, a hybrid device can be implemented to detect 740 atrial arrhythmia. It is noted that a hybrid device can perform ventricular and atrial arrhythmia detection and arrhythmic episode confirmation using one or both of the intrathoracic and transthoracic systems.

Upon declaring an atrial episode, the hybrid device can deliver 742 an appropriate therapy to the subject atrium using the intrathoracic system, it being assumed that the intrathoracic system includes an atrial lead or leads. During delivery of atrial therapy by the intrathoracic system, the transthoracic system can provide ventricular tachyarrhythmia backup therapy 744 if required. Cessation of the atrial arrhythmia can be confirmed 746 using one or both of the intrathoracic and transthoracic systems. It can be appreciated that the atrial therapy of block 742 can alternatively be delivered by the transthoracic system and that the ventricular tachyarrhythmia backup therapy of block 744 can instead be provided by the intrathoracic system.

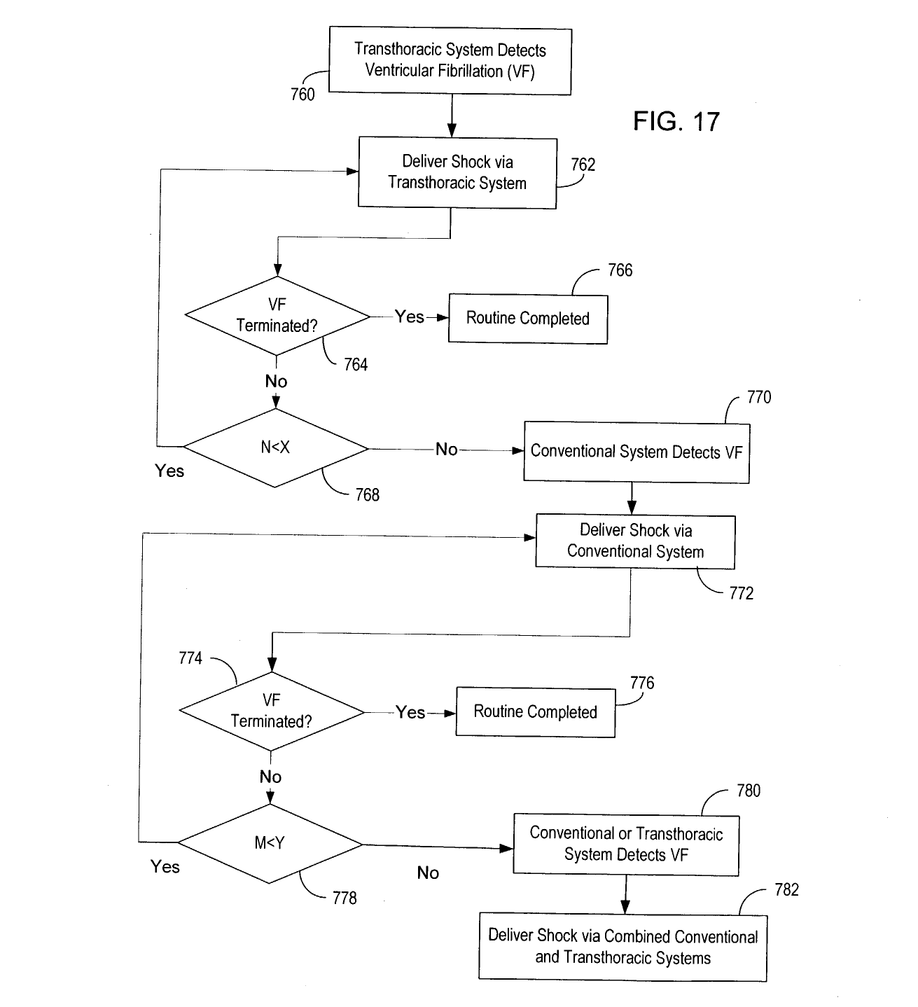

FIG. 17 illustrates various processes associated with the treatment of ventricular fibrillation (VF) using a hybrid device in accordance with an embodiment of the present invention. According to FIG. 17, counters N and M are initialized, where N represents the number of shocks delivered through the transthoracic system of the hybrid device and M represents the number of shocks delivered through the conventional system (e.g., intrathoracic system) of the hybrid device. Parameters X and Y are initialized, where X and Y represent the maximum number of shocks allowed through the transthoracic and conventional systems, respectively.

The transthoracic system of the hybrid system detects 760 and confirms a ventricular fibrillation episode. In response, the hybrid system delivers 762 a defibrillation therapy via the transthoracic system. If the ventricular fibrillation is terminated 764, the VF detection/treatment routine is competed 766.

If the ventricular fibrillation is not terminated 764 and N is less than X 768, another shock is delivered 762 via the transthoracic system. If, however, N is not less than X 768, the conventional system detects 770 the ventricular fibrillation and, if confirmed, a shock is delivered 772 via the conventional system. If the ventricular fibrillation is terminated 774, the VF detection/treatment routine is competed 776.

If the ventricular fibrillation is not terminated 774 and M is less than Y 778, another shock is delivered 772 via the conventional system. If, however, M is not less than Y 778, the conventional or transthoracic system detects 780 the ventricular fibrillation and, if detected, a shock is delivered 782 via the combined conventional and transthoracic systems.

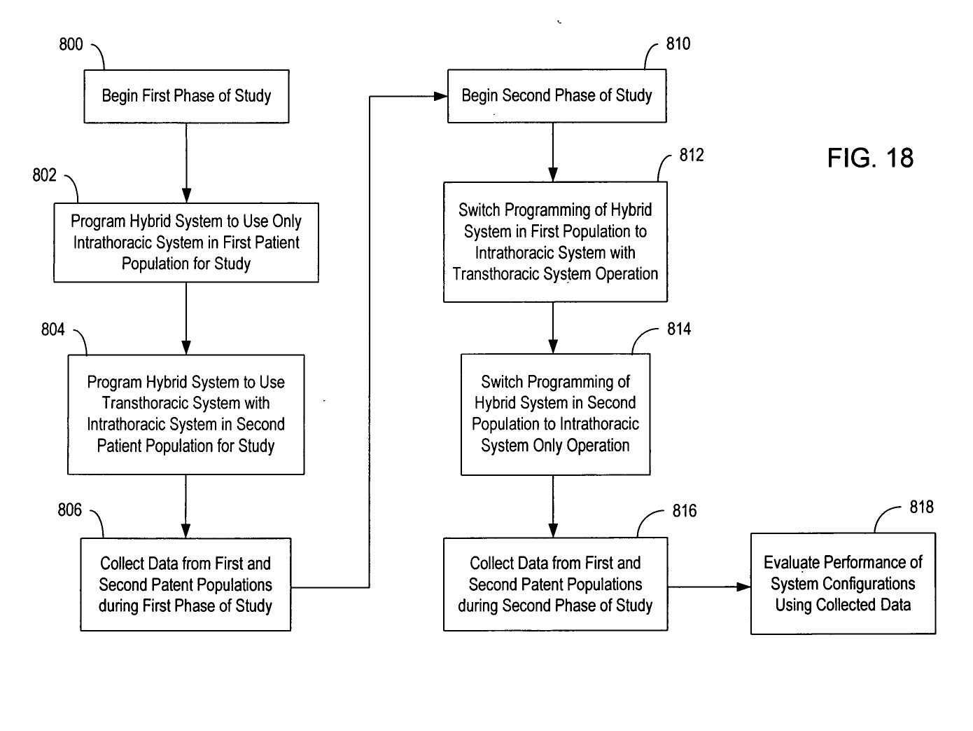

FIG. 18 illustrates a particularly useful capability involving cross-over studies conducted for a given patient population using a hybrid device of the present invention implanted in each patient of the population. As is shown in FIG. 18, a particular study involves a first phase and a second phase, which are typically, but not necessarily, equal in duration. At the beginning of the first phase 800, the hybrid systems implanted in a first patient population (e.g., a first half of the patient population) are programmed 802 such that only the intrathoracic system is operational. The hybrid systems implanted in a second patient population (e.g., a second half of the patient population) are programmed 804 such that the transthoracic systems are operative together with the intrathoracic systems. Data is collected 806 from the hybrid systems of the first and second patient populations during the first phase of the study.

At the completion of the first phase 800 and beginning of the second phase 810, the programming in the hybrid systems implanted in the first patient population switches 812 hybrid device operation from an intrathoracic-only system configuration to a configuration in which both intrathoracic and transthoracic systems are operative. The programming in the hybrid systems implanted in the second patient population switches 814 hybrid device operation from a combined intrathoracic/transthoracic system configuration to an intrathoracic-only system configuration. Data is collected 814 from the hybrid systems of the first and second patient populations during the second phase of the study. Using these data, performance of the hybrid systems in the given patient populations can be evaluated 818.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

What is claimed is:

1. An implantable system, comprising:
a housing;
energy delivery circuitry provided in the housing;
detection circuitry provided in the housing;
one or more subcutaneous electrodes coupled to the energy delivery and detection circuitry and arranged in a non-contacting relationship with respect to cardiac tissue, great vessels, and coronary vasculature;
a lead system, comprising one or more lead electrodes, coupled to the energy delivery and detection circuitry, the lead electrodes configured to contact cardiac tissue, great vessels, or coronary vasculature; and
a controller provided in the housing and coupled to the energy delivery and detection circuitry, the system configurable by the controller to operate in a standard of care configuration using at least the lead electrodes, and to operate in an alternative configuration using at least the subcutaneous electrodes, each of the standard of care and alternative system configurations respectively capable of providing cardiac activity sensing and stimulation.

2. The system according to claim 1, wherein the system is configurable by the controller to operate in the standard of care configuration using only the lead electrodes.

3. The system according to claim 1, wherein the system is configurable by the controller to operate in the alternative configuration using only the subcutaneous electrodes.

4. The system according to claim 1, wherein the system is configurable by the controller to operate in the alternative configuration using at least one of the subcutaneous electrodes and at least one of the lead electrodes.

5. The system according to claim 1, wherein the housing comprises a can electrode, the system configurable by the controller to operate in the standard of care configuration or alternative configuration using the can electrode.

6. The system according to claim 1, wherein the controller configures the system to selectively operate in one of the standard of care configuration and the alternative configuration in response to a configuration signal received from a patient-external signal source.

7. The system according to claim 1, wherein the controller configures the system to operate in one of the alternative and standard of care configurations, and, in response to a predetermined condition, configures the system to switch operation to the other of the alternative and standard of care configurations or to a combination of the alternative and standard of care configurations.

8. The system according to claim 7, wherein the predetermined condition comprises unsuccessful detection of an arrhythmia.

9. The system according to claim 7, wherein the predetermined condition comprises unsuccessful treatment of an arrhythmia.

10. The system according to claim 7, wherein the predetermined condition comprises expiration of a predetermined amount of time.

11. The system according to claim 7, wherein the predetermined condition comprises occurrence of a scheduled event.

12. The system according to claim 7, wherein the predetermined condition comprises occurrence of a predetermined number of arrhythmic episodes.

13. The system according to claim 7, wherein the predetermined condition comprises occurrence of a predetermined type of arrhythmia.

14. The system according to claim 1, wherein the controller configures the system to operate concurrently in the standard of care configuration and the alternative configuration.

15. The system according to claim 1, wherein the controller configures the system to switch operation between the standard of care configuration and the alternative configuration during an arrhythmic event.

16. The system according to claim 1, wherein the controller configures the system to switch operation between the standard of care configuration and the alternative configuration to detect an arrhythmic event.

17. The system according to claim 1, wherein the controller configures the system to switch operation between the standard of care configuration and the alternative configuration to treat an arrhythmia.

18. The system according to claim 1, wherein the controller configures the system to detect an arrhythmia through use of electrodes associated with the standard of care configuration and the alternative configuration.

19. The system according to claim 1, wherein the controller configures the system to treat an arrhythmia through use of electrodes associated with the standard of care configuration and the alternative configuration.

20. The system according to claim 1, wherein the controller:
configures the system to operate in one of the alternative and standard of care configurations to perform a first function; and
configures the system to operate in the other of the alternative and standard of care configurations to perform a second function, wherein performance of the first function enhances performance of the second function.

21. The system according to claim 20, wherein the first function comprises a first energy delivery function to instill organization in an arrhythmia, and the second function comprises a second energy delivery function to terminate the arrhythmia.

22. The system according to claim 1, wherein:
the lead system comprises an atrial lead;
the standard of care configuration provides atrial activity sensing and atrial arrhythmia therapy delivery; and
the alternative configuration provides ventricular tachyarrhythmia backup therapy for the standard of care configuration.

23. The system according to claim 1, wherein:
the lead system comprises an atrial lead having one or more atrial electrodes; and
the controller configures the system to operate in the alternative configuration to facilitate tachyarrhythmia discrimination using the subcutaneous electrodes and the one or more atrial electrodes.

24. The system according to claim 23, wherein the controller discriminates tachyarrhythmias having a ventricular origin from tachyarrhythmias having an atrial origin.

25. The system according to claim 1, wherein:
at least two of the lead electrodes are disposed in a single heart chamber; and
the standard of care configuration provides one or both of multisite sensing and multisite stimulation with respect to the single heart chamber.

26. The system according to claim 1, wherein the lead system comprises one or more transvenous electrodes.

27. The system according to claim 1, wherein the lead system comprises one or more endocardial electrodes.

28. The system according to claim 1, wherein the lead system comprises one or more epicardial electrodes.

29. The system according to claim 1, wherein the housing defines a unitary structure, and each of the subcutaneous electrodes is respectively provided on the housing.

30. The system according to claim 1, wherein at least one of the subcutaneous electrodes is provided on a rigid or shape-alterable support structure extending outwardly from the housing.

31. An implantable system, comprising:
a housing;
energy delivery circuitry provided in the housing;
detection circuitry provided in the housing;
one or more subcutaneous electrodes coupled to the energy delivery and detection circuitry and arranged in a non-contacting relationship with respect to cardiac tissue, great vessels, and coronary vasculature;
a lead system, comprising one or more lead electrodes, coupled to the energy delivery and detection circuitry, the lead electrodes configured to contact cardiac tissue, great vessels, or coronary vasculature; and
a controller provided in the housing and coupled to the energy delivery and detection circuitry, the controller configuring the system to perform a particular function when operating in each of the first and second modes and to acquire performance data associated with performance of the particular function when operating in each of the first and second modes.

32. The system according to claim 31, wherein the particular function comprises a function associated with bradycardia and tachycardia sensing.

33. The system according to claim 31, wherein the particular function comprises a function associated with tachyarrhythmia detection.

34. The system according to claim 31, wherein the particular function comprises a first sub-function associated with rate-based tachyarrhythmia detection and a second sub-function associated with morphology-based tachyarrhythmia detection.

35. The system according to claim 31, wherein the particular function comprises a function associated with one or both of stimulus waveform generation and stimulus waveform delivery.

36. The system according to claim 31, wherein the particular function comprises a function involving a configuration of one or both of the lead system and the subcutaneous electrodes.

37. The system according to claim 31, wherein the lead system comprises one or more transvenous or endocardial electrodes.

38. The system according to claim 31, wherein the lead system comprises a can electrode of the housing.

39. The system according to claim 31, wherein the lead system comprises one or more epicardial electrodes.

40. The system according to claim 31, wherein the housing defines a unitary structure, and each of the subcutaneous electrodes is respectively provided on the housing.

41. The system according to claim 31, wherein at least one of the subcutaneous electrodes is provided on a rigid or shape-alterable support structure extending outwardly from the housing.

42. An implantable system, comprising:
a housing;
energy delivery circuitry provided in the housing;
detection circuitry provided in the housing;
one or more subcutaneous electrodes coupled to the energy delivery and detection circuitry and arranged in a non-contacting relationship with respect to cardiac tissue, great vessels, and coronary vasculature;
a lead system, comprising one or more lead electrodes, coupled to the energy delivery and detection circuitry, the lead electrodes configured to contact cardiac tissue, great vessels, or coronary vasculature; and
a controller provided in the housing and coupled to the energy delivery and detection circuitry, the controller configuring the system to operate in a first mode using at least the subcutaneous electrodes, and to operate in a second mode using at least the lead electrodes.

43. The system according to claim 42, wherein the controller configures the system to operate in the second mode using only the lead electrodes.

44. The system according to claim 42, wherein the controller configures the system to operate in the second mode using the lead electrodes and the subcutaneous electrodes.

45. The system according to claim 42, wherein, in the first mode, the subcutaneous electrodes sense conditions necessitating cardiac stimulation therapy and deliver cardiac stimulation therapy in response to the sensed conditions.

46. The system according to claim 42, wherein, in the second mode, the lead electrodes sense conditions necessitating cardiac stimulation therapy and deliver cardiac stimulation therapy in response to the sensed conditions.

47. The system according to claim 42, wherein, in the second mode, the lead electrodes sense conditions necessitating cardiac stimulation therapy and the subcutaneous electrodes deliver cardiac stimulation therapy in response to the sensed conditions.

48. The system according to claim 42, wherein, in the second mode, the subcutaneous electrodes sense conditions necessitating cardiac stimulation therapy and the lead electrodes deliver cardiac stimulation therapy in response to the sensed conditions.

49. The system according to claim 42, wherein the first and second modes comprise cardioversion/defibrillation modes.

50. The system according to claim 42, wherein the first and second modes comprise pacing modes.

51. The system according to claim 42, wherein one of the first and second modes comprises a pacing mode, and the other of the first and second modes comprises a cardioversion/defibrillation mode.

52. The system according to claim 42, wherein the controller comprises memory for storing information associated with each of the first and second modes.

53. The system according to claim 42, wherein the controller comprises memory for storing system performance information acquired when operating in each of the first and second modes.

54. The system according to claim 53, wherein the controller is coupled to communication circuitry for communicating with a patient-external processing system, the processing system receiving the system performance information acquired for each of the first and second modes in real-time or in a batch mode.

55. The system according to claim 53, wherein the patient-external processing system produces comparison data using the system performance information, the comparison data comprising data indicative of system performance when operating in one of the first and second modes relative to the other of the first and second modes.

56. The system according to claim 42, wherein the controller is coupled to communication circuitry for communicating with a patient-external processing system, the processing system communicating with the controller to selectively operate the system in the first mode or the second mode.

57. The system according to claim 42, wherein the controller configures the system to operate in one of the first and second modes as a primary mode of operation, and to operate in the other of the first and second modes as a redundant mode of operation, the controller configuring the system to operate in the redundant mode in response to detection of a sensing or energy delivery deficiency while operating in the primary mode.

58. The system according to claim 42, wherein at least one of the subcutaneous electrodes defines a can electrode of the housing, and at least one other subcutaneous electrode is electrically and physically coupled to the housing via a second lead.

59. The system according to claim 42, wherein the lead system comprises one or more transvenous electrodes.

60. The system according to claim 42, wherein the lead system comprises one or more endocardial electrodes.

61. The system according to claim 42, wherein the lead system comprises one or more epicardial electrodes.

62. The system according to claim 42, wherein the housing defines a unitary structure, and each of the subcutaneous electrodes is respectively provided on the housing.

63. The system according to claim 42, wherein at least one of the subcutaneous electrodes is provided on a rigid or shape-alterable support structure extending outwardly from the housing.

64. The system according to claim 42, wherein the controller:
configures the system to operate in one of the first and second modes to perform a first function; and
configures the system to operate in the other of the first and second modes to perform a second function, wherein performance of the first function enhances performance of the second function.

65. The system according to claim 64, wherein the first function comprises a first energy delivery function to instill organization in an arrhythmia, and the second function comprises a second energy delivery function to terminate the arrhythmia.

66. The system according to claim 42, wherein:
the lead system comprises an atrial lead;
the second mode provides atrial activity sensing and atrial arrhythmia therapy delivery; and
the first mode provides backup ventricular tachyarrhythmia therapy support for the second mode.

67. The system according to claim 42, wherein:
the lead system comprises an atrial lead having one or more atrial electrodes; and
the controller configures the system to operate in the first mode to facilitate tachyarrhythmia discrimination using the subcutaneous electrodes and the one or more atrial electrodes.

68. The system according to claim 67, wherein the controller discriminates tachyarrhythmias having a ventricular origin from tachyarrhythmias having an atrial origin.

69. The system according to claim 42, wherein:
at least two of the lead electrodes are disposed in a single heart chamber; and
the second mode provides one or both of multisite sensing and multisite stimulation with respect to the single heart chamber.

70. The system according to claim 42, wherein:
at least one of the lead electrodes is disposed in each of a plurality of heart chambers; and
the second mode provides one or both of multi-chamber sensing and multi-chamber stimulation with respect to the plurality of heart chambers.

71. The system according to claim 70, wherein the system is configurable to provide resynchronization therapy.

72. The system according to claim 42, wherein the controller configures one of the first and second modes as a standard of care mode, and the other of the first and second modes as a monitoring mode for monitoring operation of the system in the standard of care mode.

73. The system according to claim 42, wherein the controller configures the first mode as a monitor-only mode, and the second mode as a treatment mode.

74. A cardiac sensing and stimulation method, comprising:
in a first mode, transthoracicly sensing cardiac activity and, in response to cardiac conditions necessitating therapy sensed while operating in the first mode, delivering cardiac stimulation therapy transthoracicly or intrathoracicly;
in a second mode, intrathoracicly sensing cardiac activity and, in response to cardiac conditions necessitating therapy sensed while operating in the second mode, intrathoracicly or transthoracicly delivering cardiac stimulation therapy; and
selectively enabling and disabling the first and second modes for independent or cooperative operation.

75. The method according to claim 74, wherein:
in the first mode, sensing cardiac activity transthoracicly or intrathoracicly, and, in response to cardiac conditions necessitating therapy sensed while operating in the first mode, delivering cardiac stimulation therapy transthoracicly; and
in the second mode, sensing cardiac activity intrathoracicly or transthoracicly and, in response to cardiac conditions necessitating therapy sensed while operating in the second mode, intrathoracicly delivering cardiac stimulation therapy.

76. The method according to claim 74, wherein one of the first and second modes is a standard of care mode, and the other of the first and second modes is a monitoring mode.

77. The method according to claim 74, wherein the first mode is a monitor-only mode, and the second mode is a treatment mode.

78. The method according to claim 74, wherein selectively enabling and disabling the first and second modes comprises enabling the first and second modes for concurrent operation.

79. The method according to claim 74, wherein selectively enabling and disabling the first and second modes comprises selectively enabling and disabling the first and second modes for sequential operation.

80. The method according to claim 74, wherein selectively enabling and disabling the first and second modes comprises selectively enabling and disabling the first and second modes for tiered operation during an arrhythmic event.

81. The method according to claim 74, wherein the first and second modes comprise cardioversion/defibrillation modes.

82. The method according to claim 74, wherein the first and second modes comprise pacing modes.

83. The method according to claim 74, wherein one of the first and second modes comprises a pacing mode, and the other of the first and second modes comprises cardioversion/defibrillation mode.

84. The method according to claim 74, further comprising selectively enabling and disabling the first and second modes from a patient-external location.

85. The method according to claim 74, further comprising storing information associated with each of the first and second modes.

86. The method according to claim 74, further comprising storing performance information acquired when operating in each of the first and second modes.

87. The method according to claim 86, further comprising transmitting the performance information to a patient-external location in real-time or in a batch mode.

88. The method according to claim 86, further comprising producing comparison data using the performance information, the comparison data comprising data indicative of performance when operating in one of the first and second modes relative to the other of the first and second modes.

89. The method according to claim 74, further comprising operating in one of the first and second modes as a primary mode of operation, and operating in the other of the first and second modes as a redundant mode of operation in response to a deficiency detected while operating in the primary mode.

90. The method according to claim 74, wherein one of the first and second modes defines a standard of care mode, and the other of the first and second modes defines a test mode.

91. The method according to claim 74, further comprising:
performing a first function while operating in one of the first and second modes; and
performing a second function while operating in the other of the first and second modes, wherein performance of the first function enhances performance of the second function.

92. The method according to claim 91, wherein the first function comprises a first energy delivery function to instill organization in an arrhythmia, and the second function comprises a second energy delivery function to terminate the arrhythmia.

93. The method according to claim 74, further comprising:
sensing atrial activity; and
delivering one or both of bradycardia pacing and antitachycardia pacing.

94. The method according to claim 74, further comprising:
intrathoracicly sensing atrial activity; and
discriminating tachyarrhythmias using sensed ventricular activity and the sensed atrial activity.

95. The method according to claim 94, further comprising discriminating tachyarrhythmias having a ventricular origin from tachyarrhythmias having an atrial origin.

96. The method according to claim 74, wherein the second mode provides one or both of multisite sensing and multisite stimulation with respect to a single heart chamber.

97. The method according to claim 74, further comprising:
intrathoracicly sensing atrial activity and, in response to conditions necessitating atrial therapy, delivering atrial stimulation therapy intrathoracicly or transthoracicly; and
providing transthoracic ventricular tachyarrhythmia backup therapy in response to conditions necessitating ventricular therapy sensed while delivering atrial stimulation therapy.

98. A cardiac sensing and stimulation method, comprising:
in a first mode, transthoracicly sensing cardiac activity and, in response to cardiac conditions necessitating therapy sensed while operating in the first mode, delivering cardiac stimulation therapy transthoracicly or intrathoracicly;
in a second mode, intrathoracicly sensing cardiac activity and, in response to cardiac conditions necessitating therapy sensed while operating in the second mode, intrathoracicly or transthoracicly delivering cardiac stimulation therapy;
performing a particular function when operating in each of the first and second modes; and
acquiring performance data associated with performance of the particular function when operating in each of the first and second modes.

99. The method according to claim 98, wherein the particular function comprises a function associated with bradycardia and tachycardia sensing.

100. The method according to claim 98, wherein the particular function comprises a function associated with bradyarrhythmia or tachyarrhythmia detection.

101. The method according to claim 98, wherein the particular function comprises a first sub-function associated with rate-based tachyarrhythmia detection and a second sub-function associated with morphology-based tachyarrhythmia detection.

102. The method according to claim 98, wherein the particular function comprises a function associated with stimulus waveform generation or stimulus waveform delivery.

103. The method according to claim 98, wherein the particular function comprises a function involving a configuration of one or both of the lead system and the subcutaneous electrodes.

104. The method according to claim 98, further comprising storing performance information associated with performance of the particular function when operating in each of the first and second modes.

105. The method according to claim 104, further comprising transmitting the performance information to a patient-external location in real-time or in a batch mode.

106. The method according to claim 104, further comprising producing comparison data using the performance information, the comparison data comprising data indicative of performance when operating in one of the first and second modes relative to the other of the first and second modes.

107. A cardiac sensing and stimulation system, comprising:
means for transthoracicly sensing cardiac activity in a first mode;
means for delivering cardiac stimulation therapy transthoracicly or intrathoracicly in response to cardiac conditions necessitating therapy sensed while operating in the first mode;
means for intrathoracicly sensing cardiac activity in a second mode;
means for intrathoracicly or transthoracicly delivering cardiac stimulation therapy in response to cardiac conditions necessitating therapy sensed while operating in the second mode; and
means for selectively enabling and disabling the first and second modes.

108. The system according to claim 107, further comprising:
means for sensing cardiac activity transthoracicly or intrathoracicly in the first mode;
means for delivering cardiac stimulation therapy transthoracicly in response to cardiac conditions necessitating therapy sensed while operating in the first mode;
means for sensing cardiac activity intrathoracicly or transthoracicly in the second mode; and
means for intrathoracicly delivering cardiac stimulation therapy in response to cardiac conditions necessitating therapy sensed while operating in the second mode.

109. The system according to claim 107, further comprising:
means for performing a first function while operating in one of the first and second modes; and
means for performing a second function while operating in the other of the first and second modes, wherein performance of the first function enhances performance of the second function.

110. A cardiac sensing and stimulation system, comprising:
means for transthoracicly sensing cardiac activity in a first mode;
means for delivering cardiac stimulation therapy transthoracicly or intrathoracicly in response to cardiac conditions necessitating therapy sensed while operating in the first mode;
means for intrathoracicly sensing cardiac activity in a second mode;
means for intrathoracicly or transthoracicly delivering cardiac stimulation therapy in response to cardiac conditions necessitating therapy sensed while operating in the second mode;
means for performing a particular function when operating in each of the first and second modes; and
means for acquiring performance data associated with performance of the particular function when operating in each of the first and second modes.

111. The method according to claim 110, further comprising:
means for storing performance information associated with performance of the particular function when operating in each of the first and second modes; and
means for transmitting the performance information to a patient-external location.
